SEQUENCE LISTING (38 sequences)

SEQ ID NO: 1 - DNA, 990 bp, Escherichia coli
  CDS (1)..(990)
atgcacaaatttactaaagccctggcagccattggtctggcagccgttatgtcacaatccgctatggcggagaacctgaagctcggttttctggtgaagcaaccggaagagccgtggttccagaccgaatggaagtttgccgataaagccgggaaggatttagggtttgaggttattaagattgccgtgccggatggcgaaaaaacattgaacgcgatcgacagcctggctgccagtggcgcaaaaggtttcgttatttgtactccggaccccaaactcggctctgccatcgtcgcgaaagcgcgtggctacgatatgaaagtcattgccgtggatgaccagtttgttaacgccaaaggtaagccaatggataccgttccgctggtgatgatggcggcgactaaaattggcgaacgtcagggccaggaactgtataaagagatgcagaaacgtggctgggatgtcaaagaaagcgcggtgatggcgattaccgccaacgaactggataccgcccgccgccgtactacgggatctatggatgcgctgaaagcggccggattcccggaaaaacaaatttatcaggtacctaccaaatctaacgacatcccgggggcatttgacgctgccaactcaatgctggttcaacatccggaagttaaacattggctgatcgtcggtatgaacgacagcaccgtgctgggcggcgtacgcgcgacggaaggtcagggctttaaagcggccgatatcatcggcattggcattaacggtgtggatgcggtgagcgaactgtctaaagcacaggcaaccggcttctacggttccctgctgccaagcccggacgtacatggctataaatccagcgaaatgctttacaactgggtagcaaaagacgttgaaccgccaaaatttaccgaagttaccgacgtggtactgatcacgcgtgacaactttaaagaagaactggagaaaaaaggtttaggcggtaagtaa

SEQ ID NO: 2 - protein, 329 aa, Escherichia coli
MHKFTKALAAIGLAAVMSQSAMAENLKLGFLVKQPEEPWFQTEWKFADKAGKDLGFEVIKIAVPDGEKTLNAIDSLAASGAKGFVICTPDPKLGSAIVAKARGYDMKVIAVDDQFVNAKGKPMDTVPLVMMAATKIGERQGQELYKEMQKRGWDVKESAVMAITANELDTARRRTTGSMDALKAAGFPEKQIYQVPTKSNDIPGAFDAANSMLVQHPEVKHWLIVGMNDSTVLGGVRATEGQGFKAADIIGIGINGVDAVSELSKAQATGFYGSLLPSPDVHGYKSSEMLYNWVAKDVEPPKFTEVTDVVLITRDNFKEELEKKGLGGK

SEQ ID NO: 3 - DNA, 1515 bp, Escherichia coli
  CDS (1)..(1515)
atgcaacagtctaccccgtatctctcatttcgcggcatcggtaaaacgtttcccggcgttaaggcgctgacggatattagttttgactgctatgccggtcaggttcatgcgttgatgggtgaaaatggcgcaggaaaatcaactctcttaaaaatcctcagcggcaactatgcgccaaccacgggttctgtagtgattaatgggcaggaaatgtccttttccgacacgaccgcagcacttaacgcgggcgtggcgattatttaccaggaactgcatctcgtgccggaaatgaccgtcgcggaaaacatctatctcggccagctgccgcataaaggcggcattgtgaatcgctcattgctgaattatgaggcgggtttacaacttaaacatcttggtatggatattgacccggacacgccgctgaaatatctctccattggtcagtggcagatggttgaaatcgccaaagcgctggcgcgtaacgccaaaattatcgcctttgatgagccaaccagctccctctctgcccgtgaaatcgacaatcttttccgcgttattcgtgaactgcgaaaagaggggcgggtaatcttatacgtttctcaccgtatggaagaaatatttgccctcagcgatgccattactgtctttaaagatggacgttatgtcaaaacctttaccgatatgcagcaggttgaccacgacgcgctggtgcaggcgatggtcgggcgcgacattggcgatatctacggctggcaaccgcgtagttatggcgaggagcgcctacgtcttgatgctgtgaaagcaccaggcgtgcgtacgccaataagtctggcggttcgcagtggtgaaattgttgggctgtttggtctggtaggggcggggcgtagcgaattaatgaaaggcatgtttggcgggacgcaaatcaccgccggtcaggtttatatcgaccaacagccgatcgatattcgtaaaccgagccacgccattgccgcaggcatgatgctctgcccggaagatcgcaaagcggaaggcattattcccgtgcactccgttcgcgacaatatcaacatcagtgccagacgtaaacatgtgctcggcggttgtgtaatcaacaacggttgggaagaaaacaatgccgatcaccacattcgttcgctcaacatcaaaacgccgggcgcggagcaactgatcatgaatctctcaggcggaaatcagcaaaaagccattctgggccgctggttatcggaagagatgaaggtcattttgctggatgaacctacgcgcggcattgatgttggcgctaagcacgaaatatataacgtaatttatgcgctggcggcgcagggcgtggcggtgctgtttgcctccagcgacttacctgaagtcctcggcgttgccgaccggattgtggtgatgcgggaaggtgaaatcgccggtgaattgttacacgagcaggcagatgagcgtcaggcactgagccttgcgatgcctaaagtcagccaggctgttgcctga

SEQ ID NO: 4 - protein, 504 aa, Escherichia coli
MQQSTPYLSFRGIGKTFPGVKALTDISFDCYAGQVHALMGENGAGKSTLLKILSGNYAPTTGSVVINGQEMSFSDTTAALNAGVAIIYQELHLVPEMTVAENIYLGQLPHKGGIVNRSLLNYEAGLQLKHLGMDIDPDTPLKYLSIGQWQMVEIAKALARNAKIIAFDEPTSSLSAREIDNLFRVIRELRKEGRVILYVSHRMEEIFALSDAITVFKDGRYVKTFTDMQQVDHDALVQAMVGRDIGDIYGWQPRSYGEERLRLDAVKAPGVRTPISLAVRSGEIVGLFGLVGAGRSELMKGMFGGTQITAGQVYIDQQPIDIRKPSHAIAAGMMLCPEDRKAEGIIPVHSVRDNINISARRKHVLGGCVINNGWEENNADHHIRSLNIKTPGAEQLIMNLSGGNQQKAILGRWLSEEMKVILLDEPTRGIDVGAKHEIYNVIYALAAQGVAVLFASSDLPEVLGVADRIVVMREGEIAGELLHEQADERQALSLAMPKVSQAVA

SEQ ID NO: 5 - DNA, 990 bp, Escherichia coli
  CDS (1)..(990)
atgatgtcttctgtttctacatcggggtctggcgcacctaagtcgtcattcagcttcgggcgtatctgggatcagtacggcatgctggtggtgtttgcggtgctctttatcgcctgtgccatttttgtcccaaattttgccaccttcattaatatgaaagggttgggcctggcaatttccatgtcggggatggtggcttgtggcatgttgttctgcctcgcttccggtgactttgacctttctgtcgcctccgtaattgcctgtgcgggtgtcaccacggcggtggttattaacctgactgaaagcctgtggattggcgtggcagcggggttgttgctgggcgttctctgtggcctggtcaatggctttgttatcgccaaactgaaaataaatgctctgatcacgacattggcaacgatgcagattgttcgaggtctggcgtacatcatttcagacggtaaagcggtcggtatcgaagatgaaagcttctttgcccttggttacgccaactggttcggtctgcctgcgccaatctggctcaccgtcgcgtgtctgattatctttggtttgctgctgaataaaaccacctttggtcgtaacaccctggcgattggcgggaacgaagaggccgcgcgtctggcgggtgtaccggttgttcgcaccaaaattattatctttgttctctcaggcctggtatcagcgatagccggaattattctggcttcacgtatgaccagtgggcagccaatgacgtcgattggttatgagctgattgttatctccgcctgcgttttaggtggcgtttctctgaaaggtggcatcggaaaaatctcatatgtggtggcgggtatcttaattttaggcaccgtggaaaacgccatgaacctgcttaatatttctcctttcgcgcagtacgtggttcgcggcttaatcctgctggcagcggtgatcttcgaccgttacaagcaaaaagcgaaacgcactgtctga

SEQ ID NO: 6 - protein, 329 aa, Escherichia coli
MMSSVSTSGSGAPKSSFSFGRIWDQYGMLVVFAVLFIACAIFVPNFATFINMKGLGLAISMSGMVACGMLFCLASGDFDLSVASVIACAGVTTAVVINLTESLWIGVAAGLLLGVLCGLVNGFVIAKLKINALITTLATMQIVRGLAYIISDGKAVGIEDESFFALGYANWFGLPAPIWLTVACLIIFGLLLNKTTFGRNTLAIGGNEEAARLAGVPVVRTKIIIFVLSGLVSAIAGIILASRMTSGQPMTSIGYELIVISACVLGGVSLKGGIGKISYVVAGILILGTVENAMNLLNISPFAQYVVRGLILLAAVIFDRYKQKAKRTV

SEQ ID NO: 7 - DNA, 60 bp, Artificial
  primer1
cacaacactaaacctataagttggggaaatacaatgtgaagcctgcttttttatactaag

SEQ ID NO: 8 - DNA, 60 bp, Artificial
  primer2
gccgatgggcgccatttttcactgcggcaagaattacgctcaagttagtataaaaaagct

SEQ ID NO: 9 - DNA, 22 bp, Artificial
  primer3
tcctggcattgattcagcctgt

SEQ ID NO: 10 - DNA, 21 bp, Artificial
  primer4
ccagcagcatgagagcgatga

SEQ ID NO: 11 - DNA, 55 bp, Artificial
  primer5
gtgcatggttctctccagctttagtgtcgttttgtgcgctcacaattccacacat

SEQ ID NO: 12 - DNA, 60 bp, Artificial
  primer6
ctgcgatgtgatattgctctcctatggagaattaatcgctcaagttagtataaaaaagct

SEQ ID NO: 13 - DNA, 18 bp, Artificial
  primer7
tggattaatctgctgtgc

SEQ ID NO: 14 - DNA, 18 bp, Artificial
  primer8
accgagcttcaggttctc

SEQ ID NO: 15 - protein, 327 aa, Yersinia pseudotuberculosis
MHKLTKALAVVGLAAVMSHSAIAESMKLGFLVKQPEEPWFQTEWKFADKAGKDLGFEVIKIAVPDGEKTLNAIDSLAASGAKGFVICTPDPKLGPAIEAKARSYNLKVIAVDDQFVNAKGKPMESVPLVMMAATKIGERQGQELWKEMNKRGWQPAETAVMAITSDELDTARRRTGGSMAALQASGFPEKQIYKVPTKSNDIPGAFDAANSMLVQHPEVKNWLIVGMNDNTVLGGVRATEGQGFKAPNVIGIGINGVDAVSELSKGQATGFYGSLLPSPDIHGYKSIQMLHDWVTKDVEPAKFTEVTDVVLITRDNFKAELEKKGLL

SEQ ID NO: 16 - protein, 327 aa, Yersinia pestis
MHKLTKALAVVGLAAVMSHSAIAESMKLGFLVKQPEEPWFQTEWKFADKAGKDLGFEVIKIAVPDGEKTLNAIDSLAASGAQGFVICTPDPKLGPAIEAKARSYNLKVIAVDDQFVNAKGKPMESVPLVMMAATKIGERQGQELWKEMNKRGWQPAETAVMAITSDELDTARRRTGGSMAALQASGFPEKQIYKVPTKSNDIPGAFDAANSMLVQHPEVKNWLIVGMNDNTVLGGVRATEGQGFKAPNVIGIGINGVDAVSELSKGQATGFYGSLLPSPDIHGYKSIQMLHDWVTKDVEPAKFTEVTDVVLITRDNFKAELEKKGLL

SEQ ID NO: 17 - protein, 327 aa, Erwinia carotovora subsp. atroseptica
MHKFTKALAAIGLAAVMSQSAMAENIKLGFLVKQPEEPWFQTEWKFADKAGKDLGFDVIKIAVPDGEKTLNAIDSLAASGAKGFVICTPDPKLGPAIIAKARSYNLKVIAVDDQFVNAKGQPMDTVPLVMMAATKIGERQGQELYKEMNKRGWKVDETGVMAITADELDTARRRTAGSMDALKAAGFPEKQIYRVPTKSNDIPGAFDAANSMLVQHPGVKNWLIIGMNDNTVLGGVRATEGQGFKAANVIGIGINGVDAVSELSKGQATGFFGSLLPSPDIHGYKSIQMLNDWVTKGVEPEKFTEVTDVVLITRDNFKVELEKKGLM

SEQ ID NO: 18 - protein, 329 aa, Shigella sonney
MHKFTKALAAIGLAAVMSQSAMAENLKLGFLVKQPEEPWFQTEWKFADKAGKDLGFEVIKIAVPDGEKTLNAIDSLAASGAKGFVICTPDPKLGSAIVAKARGYDMKVIAVDDQFVNAKGKPMDTVPLVMMAATKIGERQGQELYKEMQKRGWDVKESAVMAITANELDTARRRTTGSMDALKAAGFPEKQIYQVPTKSNDIPGAFDAANSMLVQHPEVKHWLIVGMNDSTVLGGVRATEGQGFKAADIIGIGINGVDALSELSKAQATGFYGSLLPSPDVHGYKSSEMLYNWVAKDVEPPKFTEVTDVVLITRDNFKEELEKKGLGGK

SEQ ID NO: 19 - protein, 329 aa, Shigella dysenteriae serotype 1
MHKFTKALAAIGLAAVMSQSAMAENLKLGFLVKQPEEPWFQTEWKFADKAGKDLGFEVIKIAVPDGEKTLNAIDSLAASGAKGFVICTPDPKLGSAIVAKARGYDMKVIAVDDQFVNAKGKPMDTVPLVMMAATKIGERQGQELYKEMQKRGWDVKESAVMAITSNELDTARRRTTGSMDALKAAGFPEKQIYQVPTKSNDIPGAFDAANSMLVQHPEVKHWLIVGMNDSTVLGGVRATEGQGFKAADIIGIGINGVDAVSELSKAQATGFYGSLLPSPDVHGYKSSEMLYNWVAKGVEPTKFTEVTDVVLITRDNFKEELEKKGLGGK

SEQ ID NO: 20 - protein, 331 aa, Pseudomonas mallei
MKRRTFITLAAAAAVAAAGLPAQAAEPVKIGFLVKQPEEPWFQDEWKFAELAAKDKGFTLVKIGAPSGEKVMSAIDNLAAQKAQGFIICTPDVKLGPGIVAKAKSHGLKMMTVDDRLVDGAGKPIESVPHMGISAYDIGKQVGGGIAAEIKRRGWNMNEVGAIDITYEQLPTAHDRTTGATDALVAAGFPKANVIAAPQAKTDTENAFNAANIALTKNPKFKHWVAYGLNDEAVLGAVRAAEGRGFKAADMIGIGIGGSDSALSEFKKPQPTGFFGTVIISPKRHGEETSELMYAWITQGKAPPPLTLTTGMLATRENVAQVRETMGLAAK

SEQ ID NO: 21 - protein, 331 aa, Pseudomonas solanacearum
MNQQRRGTFRAIAAATVFAIAGGFVHAAEEVKIGFLVKQPEEPWFQDEWRFADQAAKEKGFKLVKIGVPSGGEVLTAIDNLGAQHAQGFVICVPDVKLGPAVVAKARQNNLKLMTVDDRLVDGGGKPIEAVPHMGISATRIGEQVGEAIAQEMKKRGWNPSEVGAIRIAYDQLPTARERTDGAVAALAKAGFPAANVLTSPQAKTDTEAAFNAADITLTKNPRFKHWIAFGLNDEAVLGAVRAAEGHGIKAADIIGVGIGGSQSALNEFAKPEKTGFFGTVLISPKRHGYETSMNMYSWITASKAPEPLILTSGRLMTRENEKAVRQEMGL

SEQ ID NO: 22 - protein, 333 aa, Pseudomonas pseudomallei
MGLRWPQAALVCASLAAGLSAAAPAHAQGAAPVKIGFVVKQPDDPWFQDEWRFAEQAAKDKHFTLVKIAAPSGEKVSTALDSLAAQKAQGVIICAPDVKLGPGIAAKARRYGMKLMSVDDQLVDGRGAPLADVPHMGISAYRIGRQVGDAIAAEAKRRGWNPAEVGVLRLAYDQLPTARERTTGAVDALKAAGFAAANVVDAPEMTADTEGAFNAANIAFTKHRNFKHWVAFGSNDDTTVGAVRAGEGRGIGADDMIAVGINGSQVALNEFAKPKPTGFFGSILLNPRLHGYDTSVNMYDWITQNRAPPPVVLTSGTLITRANEKTARAQLGL

SEQ ID NO: 23 - protein, 533 aa, Yersinia pestis
MQPKLYKEAIMSAPHSALQAELDAAQSPYLAFRGIGKSFPGVLALDDISFTCQAGQIHALMGENGAGKSTLLKILSGNYTPTQGEIHIKGKAVNFTNTTDALDAGVAIIYQELHLVPEMTVAENIYLGQLPTKMGMVDRKLLRYESRIQLSHLGLDIDPDTPLKYLSIGQWQMVEIAKALARNAKIIAFDEPTSSLSAREIEQLFRVIRELRAEGRVILYVSHRMEEIFALSDAITVFKDGRYVRTFDDMTQVNNASLVQAMVGRNLGDIYGYQPREIGSERLTLQAVKAIGVASPISLTVHQGEIVGLFGLVGAGRSELLKGLFGDTKLTSGKLLLDGQPLTIRSPIDAISAGIMLCPEDRKADGIIPVHSVQDNINISARRKTLTAGCLINNRWEADNALLRIQSLNIKTPGPQQLIMNLSGGNQQKAILGRWLSEDMKVILLDEPTRGIDVGAKHEIYNVIYQLAKQGIAVLFASSDLPEVLGLADRIVVMREGAISGELDHEYATEEQALSLAMLRTPNIATNTASAVA

SEQ ID NO: 24 - protein, 512 aa, Yersinia pseudotuberculosis
MDAAQSPYLAFRGIGKSFPGVLALDDISFTCQAGQIHALMGENGAGKSTLLKILSGNYTPTQGEIHIKGKAVNFTNTTDALDAGVAIIYQELHLVPEMTVAENIYLGQLPTKMGMVDRKLLRYESRIQLSHLGLDIDPDTPLKYLSIGQWQMVEIAKALARNAKIIAFDEPTSSLSAREIEQLFRVIRELRAEGRVILYVSHRMEEIFALSDAITVFKDGRYVRTFDDMTQVNNASLVQAMVGRNLGDIYGYQPREIGSERLTLQAVKAIGVASPISLTVHQGEIVGLFGLVGAGRSELLKGLFGDTKLTSGKLLLDGQPLTIRSPIDAISAGIMLCPEDRKADGIIPVHSVQDNINISARRKTLTAGCLINNRWEADNALLRIQSLNIKTPGPQQLIMNLSGGNQQKAILGRWLSEDMKVILLDEPTRGIDVGAKHEIYNVIYQLAKQGIAVLFASSDLPEVLGLADRIVVMREGAISGELDHEYATEEQALSLAMLRTPNIATNTASAVA

SEQ ID NO: 25 - protein, 507 aa, Erwinia carotovora subsp. atroseptica
MTAQSPYLSFHGIGKEFPGVKALSDISFSCHAGQIHALMGENGAGKSTLLKILSGNYSPSAGEIHIQGKPVQFSNTMDALNAGVAIIYQELHLVPEMTVAENIYLGQLPHKYGMVNYSLLRYEAKLQLQHLGLDIDPDTPLKYLSIGQWQMVEIAKALARNAKIIAFDEPTSSLSAREIEQLFRVITELRSEGRIILYVSHRMEEIFALSDAITVFKDGRYVCTFDDMQQVNHESLVQAMVGRNLGNIYGYAPRPHGEDRLTLKDVKAPGVKSTISLNVKQGEIVGLFGLVGAGRSELMKGLFGATKITSGQVLLDGKPLVVNSPIDAIRQGVMLCPEDRKADGIIPVHSVRDNINISARRKSLKAGFIINNQWEADNAAQRIDALNIKTPSDEQLIMNLSGGNQQKVILGRWLSEEMKVILLDEPTRGIDVGAKHEIYHVIYELANQGIAVLFASSDLPEVLGLADRIIVMREGAVSGELLHADATEQKVLSLAMLRIPDIESAVA

SEQ ID NO: 26 - protein, 504 aa, Shigella dysenteriae serotype 1
MQQSTPYLSFRGIGKTFPGVKALTDISFDCYAGQVHALMGENGAGKSTLLKILSGNYAPTTGSVVINGQEMSFSDTTAALNAGVAIIYQELHLVPEMTVAENIYLGQLPHKGGIVNRSLLNYEAGLQLKHLGMDIDPDTPLKYLSIGQWQMVEIAKALARNAKIIAFDEPTSSLSAREIDNLFRVIRELRKEGRVILYVSHRMEEIFALSDAITVFKDGRYVRTFTDMQQVDHDALVQAMVGRDIGDIYGWQPRSYGEERLRLDAVKAPGVRTPISLAVRSGEIVGLFGLVGAGRSELMKGMFGGTQITAGQVYIDQQPIDIRKPSHAIAAGMMLCPEDRKAEGIIPVHSVRDNINISARRKHVLGGCVINNGWEENNADHHIRSLNIKTPGAEQLIMNLSGGNQQKAILGRWLSEEMKVILLDEPTRGIDVGAKHEIYNVIYALAAQGVAVLFASSDLPEVLGVADRIVVMREGEIAGELLHEQADERQALSLAMPKVSQAVA

SEQ ID NO: 27 - protein, 504 aa, Shigella sonney
MQQSTPYLSFRGIGKTFPGVKALTDISFDCYAGQVHALMGENGAGKSTLLKILSGNYAPTTGSVVINGQEMSFSDTTAALNAGVAIIYQELHLVPEMTVAENIYLGQLPHKGGIVNRSLLNYEAGLQLKHLGMDIDPDTPLKYLSIGQWQMVEIAKALARNAKIIAFDEPTSSLSAREIDNLFRVIRELRKEGRVILYVSHRMEEIFALSDAITVFKDGRYVKTFTDMQQVDHDALVQAMVGRDIGDIYGWQPRSYGEERLRLDAVKAPGVRTPISLAVRSGEIVGLFGLVGAGRSELMKGLFGGTQITAGQVYIDQQPIDIRKPSHAIAAGMMLCPEDRKAEGIIPVHSVRDNINISARRKHVLGGCVINNGWEENNADQHIRSLNIKTPGAEQLIMNLSGGNQQKAILGRWLSEEMKVILLDEPTRSIDVGAKHEIYNVIYALAAQGVAVLFASSDLPEVLGVADRIVVMREGEIAGELLHEQADERQALSLAMPKVSQAVA

SEQ ID NO: 28 - protein, 510 aa, Pseudomonas pseudomallei
MAGNGGDVAAALRFDNIGKVFPGVRALDGISFDVQAGQVHGLMGENGAGKSTLLKILGGEYQPDSGSVLVDGRAMRFPSAAASIAAGVAVIHQELQYVPDLTVAENLLLGRLPSALGWVRKRDAQRFVRERLAAMGVDLDAQAKLRRLSIAQRQMVEICKALLRNARVIALDEPTSSLSHRETEVLFKLVDDLRRDGRALIYISHRMDEIYRLCDACTIFRDGRQVASHASLANVPRETLVRQMVGREISDIYHYAPRALGDVRLSARALEGDALRAGASFDVRAGEIVGFFGLVGAGRSELMRVIYGAQRRTGGALTLDGEPLDIRSTRDAIRRGIVLCPEDRKEEGIVAHASVAENINISCRRHGLRAGLFLDRKREAETADRFIKLLKIKTPNRRQKIRFLSGGNQQKAILARWLAEPDLKVVILDEPTRGIDVGAKHEIYGVIYELAKRGCAIVMVSSELPEVLGVSDRIVVMSEGRIAGELARGEANEEAVLNLALPQGATAHAA

SEQ ID NO: 29 - protein, 503 aa, Pseudomonas mallei
MAAALRFDNIGKVFPGVRALDGISFDVQAGQVHGLMGENGAGKSTLLKILGGEYQLDSGSVLVDGRAMRFPSAAASIAAGVAVIHQELQYVPDLTVAENLLLGRLPSALGWVRKRDAQRFVRERLAAMGVDLDAQAKLRRLSIAQRQMVEICKALLRNARVIALDEPTSSLSHRETEVLFKLVDDLRRDGRALIYISHRMDEIYRLCDACTIFRDGRQVASHASLANVPRETLVRQMVGREISDIYHYAPRALGDVRLSARALEGDALRAGASFDVRAGEIVGFFGLVGAGRSELMRVIYGAQRRTGGALMLDGEPLDIRSTRDAIRRGIVLCPEDRKEEGIVAHASVAENINISCRRHGLRAGLFLDRKREAETADRFIKLLKIKTPNRRQKIRFLSGGNQQKAILARWLAEPDLKVVILDEPTRGIDVGAKHEIYGVIYELAKRGCAIVMVSSELPEVLGVSDRIVVMREGRIAGELARGEANEEAVLNLALPQGATAHAA

SEQ ID NO: 30 - protein, 511 aa, Pseudomonas solanacearum
MSAFLEFRGISKVFPGVRALSEVSFGIECGRVHGLLGENGAGKSTLLKILGGDYQPDGGQIAVEGRPVAFPNARAALAAGIAVIHQELQTVPELTVMDNLLLGHLPSRGGFIRQGEAMAWTRAQLARIGVDLDPKARLKHLSIGQRQMVEICKAILRDARVIALDEPTSSLSVRETDILFRLVKDLRAQGRALIYISHRLDEIFALCDGCTIFRDGRKVADFRSMADVTREQLVAQMVGRQIDDIFGYRPRAPGDVRLRVEGLMGPKLAEPASFAVRRGEIVGLFGLVGAGRSELARLVYGADRKTAGTVVLDGEPIRIRAVADAIRQGIVLCPEDRKEEGIIGCRSVSENINISCRRNRRPGWGGFNLFVDDRQEAKTADHYIARLRIKTPHRDQPIRLLSGGNQQKAILARWLAEDGMRVLIIDEPTRGIDVGAKNEIYQVLYELAERGVAVLMISSELPEILGVADRVLVMSEGRIAGELPRAQATEHAVLNLALRPRRDPCVAAQAA

SEQ ID NO: 31 - protein, 354 aa, Yersinia pestis
MKELIMSSVTLSSDKKNPVSTESNGGIPQPQQPQNAPTKSGLGLSRIWDSYGMLVVFAVVFIGCVIFVPNFGSFINMKGLGLAISMSGMVACGMLFCLASGDFDLSVASVIACAGVTTAVVINMTESLWIGVGAGLLLGAACGLINGFVIARLKINALITTLATMQIVRGLAYIISDGKAVGIEDERFFALGYTNWFGLPAPIWITVACLVLFGFLLNKTTFGRNTLAIGGNEDAARLAGVPVVRTKIIIFVLSGLVSAAAGIILASRMTSGQPMTSIGYELIVISACILGGVSLKGGIGKISYVIAGILILGTVENAMNLLNISPFSQYVVRGLILLAAVIFDRYKQLAKRTI

SEQ ID NO: 32 - protein, 349 aa, Yersinia pseudotuberculosis
MSSVTLSSDKKNPVSTESKGGIPQPQQPQNAPTKSGLGLSRIWDSYGMLVVFAVVFIGCVIFVPNFGSFINMKGLGLAISMSGMVACGMLFCLASGDFDLSVASVIACAGVTTAVVINMTESLWIGVGAGLLLGAACGLINGFVIARLKINALITTLATMQIVRGLAYIISDGKAVGIEDERFFALGYTNWFGLPAPIWITVACLVLFGFLLNKTTFGRNTLAIGGNEDAARLAGVPVVRTKIIIFVLSGLVSAAAGIILASRMTSGQPMTSIGYELIVISACVLGGVSLKGGIGKISYVIAGILILGTVENAMNLLNISPFSQYVVRGLILLAAVIFDRYKQLAKRTI

SEQ ID NO: 33 - protein, 328 aa, Erwinia carotovora subsp. atroseptica
MSTVTSATSEKKKNGMGLSRIWDNYGMLVVFAVLFLGCAIFVPNFASFINMKGLGLAISMSGMVACGMLFCLASGDFDLSVASIIACSGVATAVVINISESLWIGVGAGLLLGVAFGLLNGFVIARLKINALITTLATMQIARGLAYIISDGKAVGIEDERFFALGYANWLGLPAPIWITIGCMILFGLLLNKTTFGRNTLAIGGNEEAARLAGVPVVRTKIIIFALSGLVSAAAGIILASRMTSGQPMTSIGYELIVISACVLGGVSLKGGIGKISYVVAGVLILGTVENAMNLLNISPFSQYVVRGLILLAAVIFDRYKQLAKKTV

SEQ ID NO: 34 - protein, 329 aa, Shigella dysenteriae serotype 1
MMSSVSTSGSGAPKSSFSFGRIWDQYGMLVVFAVLFIACAIFVPNFATFINMKGLGLAISMSGMGACGMLFCLASGDFDLSVASVIACAGVTTAVVINLTESLWIGVAAGLLLGVLCGLVNGFVIAKLKINALITTLATMQIVRGLAYIISDGKAVGIEDESFFALGYANWFGLPAPIWLTVACLIIFGLLLNKTTFGRNTLAIGGNEEAARLAGVPVVRTKIIIFVLSGLVSAIAGIILASRMTSGQPMTSIGYELIVISACVLGGVSLKGGIGKISYVVAGILILGTVENAMNLLNISPFAQYVVRGLILLAAVIFDRYKQKAKPTV

SEQ ID NO: 35 - protein, 329 aa, Shigella sonney
MMSSVSTSGSGAPKSSFSFGRIWDQYGMLVVFAVLFIACAIFVPNFATFINMKGLGLAISMSGMVACGMLFCLASGDFDLSVASVIACAGVTTAVVINLTESLWIGVAAGLLLGILCGLVNGFVIAKLKINALITTLATMQIVRGLAYIISDGKAVGIEDESFFALGYANWFGLPAPIWLTVACLIIFGLLLNKTTFGRNTLAIGGNEEAARLAGVPVVRTKIIIFVLSGLVSAIAGIILASRMTSGQPMTSIGYELIVISACVLGGVSLKGGIGKISYVVAGILILGTVENAMNLLNISPFAQYVVRGLILLAAVIFDRYKQKAKRIV

SEQ ID NO: 36 - protein, 334 aa, Pseudomonas pseudomallei
MQARENLPPAAAHAAAVPTEDRQRWRQHAADYSLVAIFAAMFVAMSLTVDHFFSIDNMLGLALSISQIGMVACTMMFCLASRDFDLSIGSTVAFAGVLCAMVLNATDNTFVAIAAAVAAGAVIGFVNGAVIAYLRINALITTLATMEIVRGLGFIVSKGQAVGVSSETFIALGGLTFFGVSLPIWVTLACFVVFGVLLNQTVYGRNTLAIGGNPEASRLAGINVERTRVYIFLIQGAVTALAGVILASRITSGQPNAAQGFELNVISACVLGGVSLAGGRASISGVVIGVLIMGTVENVMNLLNIDAFYQYLVRGAILLAAVLLDQLKNRGARD

SEQ ID NO: 37 - protein, 334 aa, Pseudomonas mallei; identical to SEQ ID NO: 36

SEQ ID NO: 38 - protein, 335 aa, Pseudomonas solanacearum
MSQSQPLQRADGFAASARSAMNNTRLLRRLDDFSLPLIFAILFAALSLSVEYFFSWQNMVGLALSVSQIGMVACTMMFCLASRDFDLSIGSTVAFAGVLCATVINATGSIALGIGASLLAGAVIGGINGFVIARLKINALITTLATMEIVRGLAFIASHGQAVGVSEMAFFDLGNTIVLGVPTPVWVAALCFVAFGVLLNKTVYGRNTLAIGGNPEAARLAGVNVNLTRIVIFLVQGVIAALAGVILAARITSGQPNAAQGFELNVISACVLGGVSLMGGRASISGVLVGVLIMGTVQNAMNLLNIDAFYQYLVRGGILLAAVLVDQIKHRGGRD

(19) United States
(12) Patent Application Publication
(10) Pub. No.: US 2009/0155861 A1
(43) Pub. Date: Jun. 18, 2009

(54) METHOD FOR PRODUCING AN L-AMINO ACID USING A BACTERIUM OF THE ENTEROBACTERIACEAE FAMILY

(76) Inventors: Konstantin Vyacheslavovich Rybak, Moscow (RU); Ekaterina Aleksandrovna Slivinskaya, Moscow (RU); Marina Evgenievna Sheremet'eva, Moscow (RU); Vitaly Grigorievich Paraskevov, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 12/275,842

(22) Filed: Nov. 21, 2008

Related U.S. Application Data
(63) Continuation of application No. PCT/JP2007/060935, filed on May 23, 2007.
(60) Provisional application No. 60/867,151, filed on Nov. 24, 2006.

(30) Foreign Application Priority Data
May 23, 2006 (RU) 2006117420

(51) Int. Cl. C12P 13/08; C12N 1/21; C12P 13/04

(52) U.S. Cl. 435/115; 435/252.3; 435/252.33; 435/106

(57) ABSTRACT

A method is described for producing an L-amino acid, for example L-threonine, L-lysine, L-leucine, L-histidine, L-cysteine, L-phenylalanine, L-arginine, L-tryptophan, L-glutamic acid, L-valine, and L-isoleucine, by fermentation of glucose using a bacterium of the Enterobacteriaceae family, wherein the bacterium has been modified to enhance the activity of the high-affinity arabinose transporter coded by the araFGH operon.

This application is a continuation under 35 U.S.C. §120 to PCT Patent Application No. PCT/JP2007/060935, filed on May 23, 2007, which claims priority under 35 U.S.C. §119 to Russian Patent Application No. 2006117420, filed on May 23, 2006 and U.S. Provisional Patent Application No. 60/867,151, filed on Nov. 24, 2006, the entireties of which are incorporated by reference. The Sequence Listing filed herewith in electronic format is also hereby incorporated by reference in its entirety (File Name: US-284 Seq List; File Size: 106 KB; Date Created: 2008).

### BACKGROUND OF THE INVENTION

1. Field of the Invention

The present invention relates to a method for producing an L-amino acid such as L-threonine, L-lysine, L-leucine, L-histidine, L-cysteine, L-phenylalanine, L-arginine, L-tryptophan, L-glutamic acid, L-valine, and L-isoleucine by fermentation.

2. Brief Description of the Related Art

Conventionally, L-amino acids are industrially produced by fermentation methods utilizing strains of microorganisms obtained from natural sources, or mutants thereof. Typically, the microorganisms are modified to enhance production yields of L-amino acids. Many techniques to enhance L-amino acid production yields have been reported, including transformation of microorganisms with recombinant DNA (see, for example, U.S. Pat. No. 4,278,765). Other techniques for enhancing production yields include increasing the activities of enzymes involved in amino acid biosynthesis and/or desensitizing the target enzymes of the feedback inhibition by the resulting L-amino acid (see, for example, WO 95/16042 or U.S. Pat. Nos. 4,346,170, 5,661,012 and 6,040,160).

Strains useful in production of L-threonine by fermentation are known, including strains with increased activities of enzymes involved in L-threonine biosynthesis (U.S. Pat. Nos. 5,175,107; 5,661,012; 5,705,371; 5,939,307; EP 0219027), strains resistant to chemicals such as L-threonine and its analogs (WO 01/14525A1, EP 301572 A2, U.S. Pat. No. 5,376,538), strains with target enzymes desensitized to feedback inhibition by the produced L-amino acid or its by-products (U.S. Pat. Nos. 5,175,107; 5,661,012), and strains with inactivated threonine degradation enzymes (U.S. Pat. Nos. 5,939,307; 6,297,031).

The known threonine-producing strain Escherichia coli VKPM B-3996 (U.S. Pat. Nos. 5,175,107 and 5,705,371) is presently one of the best known threonine producers. To construct the VKPM B-3996 strain, several mutations and a plasmid, described below, were introduced into the parent strain E. coli K-12 (VKPM B-7). A mutant thrA gene (mutation thrA442) encodes aspartokinase homoserine dehydrogenase I, which is resistant to feedback inhibition by threonine. A mutant ilvA gene (mutation ilvA442) encodes threonine deaminase which has decreased activity, and results in a decreased rate of isoleucine biosynthesis and a leaky phenotype of isoleucine starvation. In bacteria containing the ilvA442 mutation, transcription of the thrABC operon is not repressed by isoleucine; and therefore, this mutation results in very efficient threonine production. Inactivation of the tdh gene encoding threonine dehydrogenase results in the prevention of threonine degradation. The genetic determinant of saccharose assimilation (scrKYABR genes) was transferred to this strain. To increase expression of the genes controlling threonine biosynthesis, the plasmid pVIC40 containing the mutant threonine operon thrA442BC was introduced into the intermediate strain TDH6. The amount of L-threonine which accumulates during fermentation of the strain can be up to 85 g/l.

By optimizing the main biosynthetic pathway of a desired compound, further improvement of L-amino acid producing strains can be accomplished via supplementation of the bacterium with increasing amounts of sugars as a carbon source, for example, glucose or arabinose. Despite the efficiency of glucose transport by PTS, access to the carbon source in a highly productive strain still may be insufficient.

It is known that the active transport of sugars and other metabolites into bacterial cells is accomplished by several different transport systems.

Among these, there are two inducible transport systems for L-arabinose utilization. The low-affinity permease (K_{M} about 0.1 mM) is encoded by the araE gene at min 61.3 and the high-affinity system (K_{M}; 1 to 3 mM) is specified by the araFGH operon at min 44.8. The araF gene encodes a periplasmic binding protein (306 amino acids) with chemotactic receptor function and the araG locus encodes at least one inner membrane protein. Both high- and low-affinity transports are under the control of the araC gene product and are thus part of the ara regulon (Escherichia coli and Salmonella, Second Edition, Editor in Chief: F. C. Neidhardt, ASM Press, Washington D.C., 1996).

The araFGH operon is the “high-affinity” L-arabinose transport operon. This operon encodes three proteins. The first is a 33,000 Mr protein that is the product of the promoter-proximal L-arabinose binding protein coding sequence, araF. A 52,000 Mr protein is encoded by araG which is downstream of araF. A 31,000 Mr protein is encoded by araH which is downstream of araG. Both of the products of the araG and araH genes are localized in the membrane fraction of the cell, implying a role in the membrane-associated complex of the high-affinity L-arabinose transport system (Horazdovsky, B. F. and Hogg, R. W., J. Mol. Biol; 197(1):27-35 (1987)).

Expression plasmids containing various portions of araFGH operon sequences were assayed for their ability to facilitate the high-affinity L-arabinose transport process in a strain lacking the chromosomal copy of this operon. Accumulation studies demonstrated that the specific induction of all three genes was necessary to restore high-affinity L-arabinose transport. Kinetic analysis of this genetically reconstituted transport system indicated that it functions with essentially wild-type parameters. Therefore, L-arabinose-binding protein-mediated transport appears to require only two inducible membrane-associated components (araG and araH) in addition to the binding protein (araF) (Horazdovsky, B. F. and Hogg, R. W., J. Bacteriol; 171(6):3053-9 (1989)).

However, there have been no reports to date of using a bacterium of the Enterobacteriaceae family with enhanced expression of the araFGH operon for the purpose of increasing the production of L-amino acids by fermentation of glucose.

### SUMMARY OF THE INVENTION

Aspects of the present invention include enhancing the productivity of L-amino acid-producing strains and providing a method for producing L-amino acids using these strains.

The above aspects were achieved by finding that enhancing the expression of the araFGH operon encoding the L-arabinose transporter can increase production of L-amino acids, such as L-threonine, L-lysine, L-leucine, L-histidine, L-cysteine, L-phenylalanine, L-arginine, L-tryptophan, L-glutamic acid, L-valine, and L-isoleucine, by fermentation using glucose as a carbon source. The insufficient access to the carbon source was simulated by deleting the PTS transport system (ptsHI-crr) in the L-amino acid producing strain.

It is an aspect of the present invention to provide an L-amino acid producing bacterium of the Enterobacteriaceae family, wherein said bacterium has been modified to enhance the expression of the araFGH operon.

It is a further aspect of the present invention to provide the bacterium described above, wherein the expression of the araFGH operon is enhanced by modifying an expression control sequence of the araFGH operon so that the gene expression is enhanced, or by increasing the copy number of the araFGH operon.

It is a further aspect of the present invention to provide the bacterium described above, wherein said bacterium is selected from the group consisting of the genera Escherichia, Enterobacter, Erwinia, Klebsiella, Pantoea, Providencia, Salmonella, Serratia, Shigella, and Morganella.

It is a further aspect of the present invention to provide the bacterium described above, wherein said operon encodes:

(A) a protein comprising the amino acid sequence of SEQ ID NO: 2 or a variant thereof;

(B) a protein comprising the amino acid sequence of SEQ ID NO: 4 or a variant thereof; and

(C) a protein comprising the amino acid sequence of SEQ ID NO: 6 or a variant thereof;

wherein said variants have the activity of the high-affinity L-arabinose transporter when said variants are combined together.

It is a further aspect of the present invention to provide the bacterium described above, wherein said operon comprises:

(A) a DNA comprising the nucleotide sequence of nucleotides 1 to 990 in SEQ ID NO: 1, or a DNA which is able to hybridize to a sequence complementary to said sequence, or a probe prepared from said sequence under stringent conditions;

(B) a DNA comprising the nucleotide sequence of nucleotides 1 to 1515 in SEQ ID NO: 3, or a DNA which is able to hybridize to a sequence complementary to said sequence, or a probe prepared from said sequence under stringent conditions; and

(C) a DNA comprising the nucleotide sequence of nucleotides 1 to 990 in SEQ ID NO: 5, or a DNA which is able to hybridize to a sequence complementary to said sequence, or a probe prepared from said sequence under stringent conditions; and

wherein, said DNAs encode proteins which have an activity of the high-affinity L-arabinose transporter when said proteins are combined together.

It is a further aspect of the present invention to provide the bacterium described above, wherein said stringent conditions comprise washing at 60° C. at a salt concentration of 1×SSC and 0.1% SDS, for approximately 15 minutes.

It is a further aspect of the present invention to provide the bacterium described above, wherein said bacterium has been additionally modified to enhance the activity of glucokinase.

It is a further aspect of the present invention to provide the bacterium described above, wherein said bacterium has been additionally modified to enhance the activity of xylose isomerase.

It is a further aspect of the present invention to provide the bacterium described above, wherein said bacterium is an L-threonine producing bacterium.

It is a further aspect of the present invention to provide the bacterium described above, wherein said bacterium has been additionally modified to enhance expression of a gene selected from the group consisting of:
the mutant thrA gene which codes for aspartokinase homoserine dehydrogenase I and is resistant to feedback inhibition by threonine;
the thrB gene which codes for homoserine kinase;
the thrC gene which codes for threonine synthase;
the rhtA gene which codes for a putative transmembrane protein;
the asd gene which codes for aspartate-β-semialdehyde dehydrogenase;
the aspC gene which codes for aspartate aminotransferase (aspartate transaminase); and
combinations thereof.

It is a further aspect of the present invention to provide the bacterium described above, wherein said bacterium is an L-lysine producing bacterium.

It is a further aspect of the present invention to provide the bacterium described above, wherein said bacterium is an L-histidine producing bacterium.

It is a further aspect of the present invention to provide the bacterium described above, wherein said bacterium is an L-phenylalanine producing bacterium.

It is a further aspect of the present invention to provide the bacterium described above, wherein said bacterium is an L-arginine producing bacterium.

It is a further aspect of the present invention to provide the bacterium described above, wherein said bacterium is an L-tryptophan producing bacterium.

It is a further aspect of the present invention to provide the bacterium described above, wherein said bacterium is an L-glutamic acid producing bacterium.

It is a further aspect of the present invention to provide a method for producing an L-amino acid comprising cultivating the bacterium described above in a culture medium which contains glucose as a carbon source, and isolating the L-amino acid from the culture medium.

It is a further aspect of the present invention to provide the method described above, wherein said L-amino acid is L-threonine.

It is a further aspect of the present invention to provide the method described above, wherein said L-amino acid is L-lysine.

It is a further aspect of the present invention to provide the method described above, wherein said L-amino acid is L-histidine.

It is a further aspect of the present invention to provide the method described above, wherein said L-amino acid is L-phenylalanine.

It is a further aspect of the present invention to provide the method described above, wherein said L-amino acid is L-arginine.

It is a further aspect of the present invention to provide the method described above, wherein said L-amino acid is L-tryptophan.

It is a further aspect of the present invention to provide the method described above, wherein said L-amino acid is L-glutamic acid.

### BRIEF DESCRIPTION OF THE DRAWINGS

FIG. 1 shows the relative positions of primers P1 and P2 on plasmid pMW118-attL-Cm-attR.

FIG. 2 shows construction of a chromosomal DNA fragment which includes the inactivated ptsHI-crr operon.

FIG. 3 shows substitution of the native promoter region of the araFGH operon in E. coli with the hybrid P_{L-tac} promoter.

FIG. 4 shows the influence of P_{L-tac}araFGH on growth of the PTS⁻ strain. In the Figure, MG1655 means E. coli strain MG1655; MGΔpts means E. coli strain MG1655ΔptsHI-crr; and MGΔpts-P-araFGH means E. coli strain MG1655 ΔptsHI-crr P_{L-tac}araFGH.

FIG. 5 shows the alignment of the primary sequences of the AraF from Escherichia coli (ECO, SEQ ID NO: 2), Shigella dysenteriae serotype 1 (SHD, SEQ ID NO: 19), Shigella sonney (SHS, SEQ ID NO: 18), Erwinia carotovora subsp. atroseptica(ERC, SEQ ID NO: 17), Yersinia pestis(YPE, SEQ ID NO: 16), Yersinia pseudotuberculosis(YPS, SEQ ID NO: 15), Pseudomonas pseudomallei (PSP, SEQ ID NO: 22), Pseudomonas mallei(PSM, SEQ ID NO:20), Pseudomonas solanacearum(PSS, SEQ ID NO:21). The alignment was done by using the PIR Multiple Alignment program (http://pir.georgetown.edu). The identical amino acids are marked by asterisk (*), similar amino acids are marked by colon (:).

FIG. 6 shows the alignment of the primary sequences of the AraG from Escherichia coli (ECO, SEQ ID NO: 4), Shigella dysenteriae serotype 1 (SHD, SEQ ID NO:26), Shigella sonney (SHS, SEQ ID NO: 27), Erwinia carotovora subsp. atroseptica (ERC, SEQ ID NO: 25), Yersinia pestis (YPE, SEQ ID NO: 23), Yersinia pseudotuberculosis (YPS, SEQ ID NO: 24), Pseudomonas pseudomallei (PSP, SEQ ID NO: 28), Pseudomonas mallei (PSM, SEQ ID NO: 29), Pseudomonas solanacearum (PSS, SEQ ID NO: 30). The alignment was done by using the PIR Multiple Alignment program (http://pir.georgetown.edu). The identical amino acids are marked by asterisk (*), similar amino acids are marked by colon (:).

FIG. 7 shows the alignment of the primary sequences of the AraH from Escherichia coli (ECO, SEQ ID NO: 6), Shigella dysenteriae serotype 1 (SHD, SEQ ID NO: 34), Shigella sonney (SHS, SEQ ID NO: 35), Erwinia carotovora subsp. atroseptica(ERC, SEQ ID NO: 33), Yersinia pestis(YPE, SEQ ID NO: 31), Yersinia pseudotuberculosis(YPS, SEQ ID NO: 32), Pseudomonas pseudomallei (PSP, SEQ ID NO: 36), Pseudomonas mallei (PSM, SEQ ID NO: 37), Pseudomonas solanacearum (PSS, SEQ ID NO: 38). The alignment was done by using the PIR Multiple Alignment program (http://pir.georgetown.edu). The identical amino acids are marked by asterisk (*), similar amino acids are marked by colon (:).

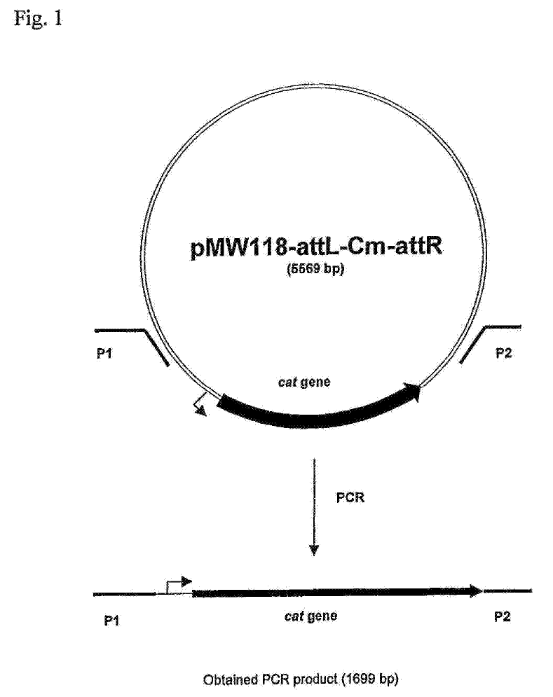

### DETAILED DESCRIPTION OF THE PREFERRED EMBODIMENTS

“L-amino acid-producing bacterium” means a bacterium which has an ability to cause accumulation of an L-amino acid in a medium when the bacterium is cultured in the medium. The L-amino acid-producing ability may be imparted or enhanced by breeding. The phrase “L-amino acid-producing bacterium” also can mean a bacterium which is able to produce and cause accumulation of an L-amino acid in a culture medium in an amount larger than a wild-type or parental strain of the bacterium, for example, E. coli, such as E. coli K-12, and preferably means that the bacterium is able to cause accumulation in a medium of an amount not less than 0.5 g/L, more preferably not less than 1.0 g/L of the target L-amino acid. The term “L-amino acids” includes L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine. L-threonine, L-lysine, L-histidine, L-phenylalanine, L-arginine, L-tryptophan, and L-glutamic acid are particularly preferred.

The Enterobacteriaceae family includes bacteria belonging to the genera Escherichia, Enterobacter, Erwinia, Klebsiella, Pantoea, Providencia, Salmonella, Serratia, Shigella, Morganella, etc. Specifically, those classified into the Enterobacteriaceae according to the taxonomy used in the NCBI (National Center for Biotechnology Information) database (http://www.ncbi.nlm.nih.gov/Taxonomy/Browser/wwwtax.cgi?id=91347) can be used. A bacterium belonging to the genus Escherichia or Pantoea is preferred.

The phrase “a bacterium belonging to the genus Escherichia” means that the bacterium is classified into the genus Escherichia according to the classification known to a person skilled in the art of microbiology. Examples of a bacterium belonging to the genus Escherichia as used in the present invention include, but are not limited to, Escherichia coli (E. coli).

The bacterium belonging to the genus Escherichia that can be used is not particularly limited; however, for example, bacteria described by Neidhardt, F. C. et al. (Escherichia coli and Salmonella typhimurium, American Society for Microbiology, Washington D.C., 1208, Table 1) are encompassed by the present invention.

The phrase “a bacterium belonging to the genus Pantoea” means that the bacterium is classified into the genus Pantoea according to the classification known to a person skilled in the art of microbiology. Some species of Enterobacter agglomerans have been recently re-classified into Pantoea agglomerans, Pantoea ananatis, Pantoea stewartii, or the like, based on the nucleotide sequence analysis of 16S rRNA etc. (Int. J. Syst. Bacteriol., 43, 162-173 (1993)).

The bacterium encompasses a strain of the Enterobacteriaceae family which has an ability to produce an L-amino acid and has been modified to enhance the expression of the araFGH operon. In addition, the bacterium of the present invention encompasses a strain of the Enterobacteriaceae family which has an ability to produce an L-amino acid and has been transformed with a DNA fragment encoding the araFGH operon so that components of the L-arabinose transporter encoded by the DNA fragment are expressed.

The phrase “activity of high-affinity L-arabinose transporter” means an activity of transporting sugars, such as L-arabinose and glucose, into the cell. The activity of the high-affinity L-arabinose transporter can be detected and measured by using membrane vesicles as described by Daruwalla et al (Biochem J., 200(3), 611-27 (1981)) or by complementation of high-affinity arabinose transport in an araFGH knockout strain (Horazdovsky, B. F. and Hogg, R. W., J. Bacteriol; 171(6):3053-9 (1989)).

The phrase “enhance the expression of the operon” means that the expression of the operon is increased compared to that of a non-modified strain, for example, a wild-type strain. Examples of such modifications include increasing the copy number of the operon(s) per cell, increasing the expression level of the operon(s), and so forth. The quantity of the copy number of the operon is measured, for example, by Southern blotting using a probe based on the operon sequence, fluorescence in situ hybridization (FISH), and the like. The level of operon expression can be measured by various known methods including Northern blotting, quantitative RT-PCR, and the like. Furthermore, wild-type strains that can act as a control include, for example, Escherichia coli K-12 or Pantoea ananatis FERM BP-6614 (WO2004099426, AU2004236516A1). Pantoea ananatis FERM BP-6614 was deposited at the National Institute of Bioscience and Human-Technology, Agency of Industrial Science and Technology, Ministry of International Trade and Industry (currently, International Patent Organism Depositary, National Institute of Advanced Industrial Science and Technology, Tsukuba Central 6, 1-1, Higashi 1-Chome, Tsukuba-shi, Ibaraki-ken, 305-8566, Japan) on Feb. 19, 1998 and received an accession number of FERM P-16644. It was then converted to an international deposit under the provisions of Budapest Treaty on Jan. 11, 1999 and received an accession number of FERM BP-6614. Although this strain was identified as Enterobacter agglomerans when it was isolated, it has been re-classified into Pantoea ananatis based on nucleotide sequence analysis of 16S rRNA etc. as described above. As a result of enhancing the intracellular activity of L-arabinose transporter, increased levels of various L-amino acids, for example, L-threonine, L-lysine, L-histidine, L-phenylalanine, L-tryptophan, or L-glutamic acid in a medium is observed.

The araFGH operon includes three genes in the following order. The araF gene (synonyms—ECK1899, b1901) encodes the L-arabinose-binding protein (synonym—B1901). The araF gene (nucleotides complementary to nucleotides 1,983,163 to 1,984,152 in the sequence of GenBank accession NC_{—}000913, gi: 16129851) is located between the yecI and araG genes on the chromosome of E. coli K-12. The araG gene (synonyms—ECK1898, b1900) encodes the ATP-binding component of the L-arabinose transporter (synonym—B1900). The araG gene (nucleotides complementary to nucleotides 1,981,579 to 1,983,093 in the sequence of GenBank accession NC_{—}000913, gi: 16129850) is located between the araF and araG genes on the chromosome of E. coli K-12. The araH gene (synonyms—ECK1897, b4460, G8206) encodes the L-arabinose-binding protein (synonym—B4460). The araH gene (nucleotides complementary to nucleotides 1,980,578 to 1,981,567 in the sequence of GenBank accession NC_{—}000913, gi: 49176167) is located between the araG and ots genes on the chromosome of E. coli K-12. araFGH operons from the following microorganisms have also been elucidated: Shigella dysenteriae serotype 1, Shigella sonney, Erwinia carotovora subsp. atroseptica, Yersinia pestis, Yersinia pseudotuberculosis, Pseudomonas pseudomallei, Pseudomonas mallei, Pseudomonas solanacearum. Examples of the araF, araG, and araH genes from Escherichia coli are represented by SEQ ID NO: 1, SEQ ID NO: 3, and SEQ ID NO: 5, respectively. The amino acid sequences encoded by the araF, araG, and araH genes are presented by SEQ ID NO: 2, SEQ ID NO: 4, and SEQ ID NO: 6, respectively.

Upon being transported into the cell, glucose is phosphorylated by glucokinase, which is encoded by the glk gene. So, it is also desirable to modify the bacterium to have enhanced activity of glucokinase. The glk gene which encodes glucokinase of Escherichia coli has been elucidated (nucleotide numbers 2506481 to 2507446 in the sequence of GenBank accession NC_{—}000913.1, gi:16127994). The glk gene is located between the b2387 and the b2389 ORFs on the chromosome of E. coli K-12.

Under appropriate conditions, xylose isomerase encoded by the xylA gene also efficiently catalyzes the conversion of D-glucose to D-fructose (Wovcha, M. G. et al, Appl Environ Microbiol. 45(4): 1402-4 (1983)). So, it is also desirable to modify the bacterium to have an enhanced activity of xylose isomerase. The xylA gene which encodes xylose isomerase of Escherichia coli has been elucidated (nucleotide numbers 3728788 to 3727466 in the sequence of GenBank accession NC_{—}000913.2, gi: 49175990). The xylA gene is located between the xylB and xylF genes on the chromosome of E. coli K-12.

The araFGH, glk and xylA genes can be obtained by PCR (polymerase chain reaction; refer to White, T. J. et al., Trends Genet., 5, 185 (1989)) utilizing primers prepared based on the known nucleotide sequences of the genes. Genes coding for L-arabinose permease from other microorganisms can be obtained in a similar manner.

The araFGH operon derived from Escherichia coli is exemplified by a DNA which encodes the following proteins:

(A) a protein comprising the amino acid sequence of SEQ ID NO: 2 or a variant thereof;

(B) a protein comprising the amino acid sequence of SEQ ID NO: 4 or a variant thereof; and

(C) a protein comprising the amino acid sequence of SEQ ID NO: 6 or a variant thereof.

The phrase “variant protein” means a protein which has changes in the sequence, whether they are deletions, insertions, additions, or substitutions of amino acids, but still maintains the desired activity at a useful level, for example, useful for the enhanced production of an L-amino acid. The number of changes in the variant protein depends on the position in the three dimensional structure of the protein or the type of amino acid residues. The number of changes may be 1 to 30, preferably 1 to 15, and more preferably 1 to 5 for the proteins shown as SEQ ID NO:2, SEQ ID NO:4, or SEQ ID NO: 6. These changes in the variants can occur in regions of the protein which are not critical for the function of the protein. This is because some amino acids have high homology to one another so the three dimensional structure or activity is not affected by such a change. These changes in the variant protein can occur in regions of the protein which are not critical for the function of the protein. Therefore, the protein variants may have a homology of not less than 70%, preferably not less than 80%, and more preferably not less than 90%, and most preferably not less than 95% with respect to the entire amino acid sequences shown in any of SEQ ID NO. 2, SEQ ID NO. 4 and SEQ ID NO. 6 as long as the activity of L-arabinose transporter is maintained when combined with the corresponding components of the high-affinity L-arabinose transporter. For example, the components of the high-affinity L-arabinose transporter may be combined as follows: a variant of the protein shown in SEQ ID NO: 2 is combined with the proteins having the amino acid sequences of SEQ ID NO: 4 and SEQ ID NO: 6, a variant of protein shown in SEQ ID NO: 4 is combined with the proteins having the amino acid sequences of SEQ ID NO: 2 and SEQ ID NO: 6, and a variant of the protein shown in SEQ ID NO: 6 is combined with proteins having the amino acid sequences of SEQ ID NO: 2 and SEQ ID NO: 4. Homology between two amino acid sequences can be determined using the well-known methods, for example, the computer program BLAST 2.0, which calculates three parameters: score, identity and similarity.

The substitution, deletion, insertion, or addition of one or several amino acid residues should be conservative mutation(s) so that the activity is maintained. The representative conservative mutation is a conservative substitution. Examples of conservative substitutions include substitution of Ser or Thr for Ala, substitution of Gln, His or Lys for Arg, substitution of Glu, Gln, Lys, His or Asp for Asn, substitution of Asn, Glu or Gln for Asp, substitution of Ser or Ala for Cys, substitution of Asn, Glu, Lys, His, Asp or Arg for Gln, substitution of Asn, Gln, Lys or Asp for Glu, substitution of Pro for Gly, substitution of Asn, Lys, Gln, Arg or Tyr for His, substitution of Leu, Met, Val or Phe for Ile, substitution of Ile, Met, Val or Phe for Leu, substitution of Asn, Glu, Gln, His or Arg for Lys, substitution of Ile, Leu, Val or Phe for Met, substitution of Trp, Tyr, Met, Ile or Leu for Phe, substitution of Thr or Ala for Ser, substitution of Ser or Ala for Thr, substitution of Phe or Tyr for Trp, substitution of His, Phe or Trp for Tyr, and substitution of Met, Ile or Leu for Val.

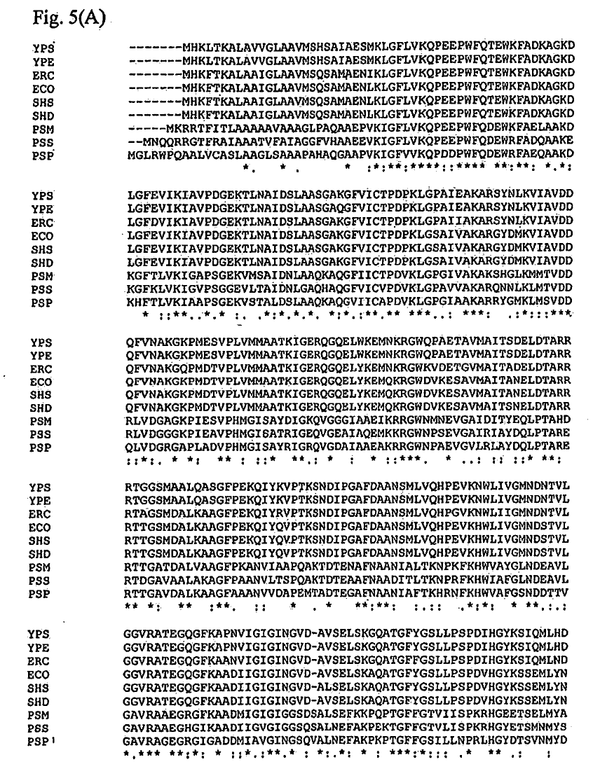

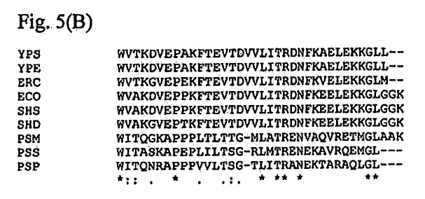

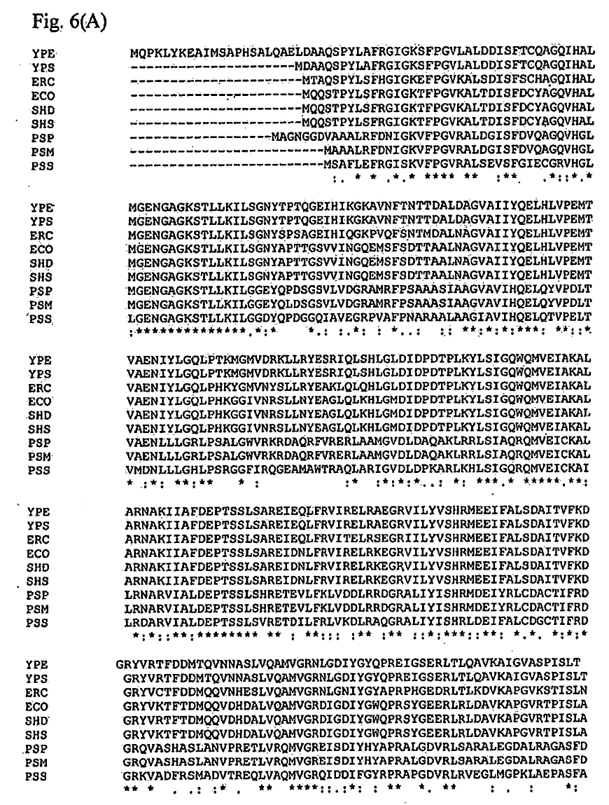

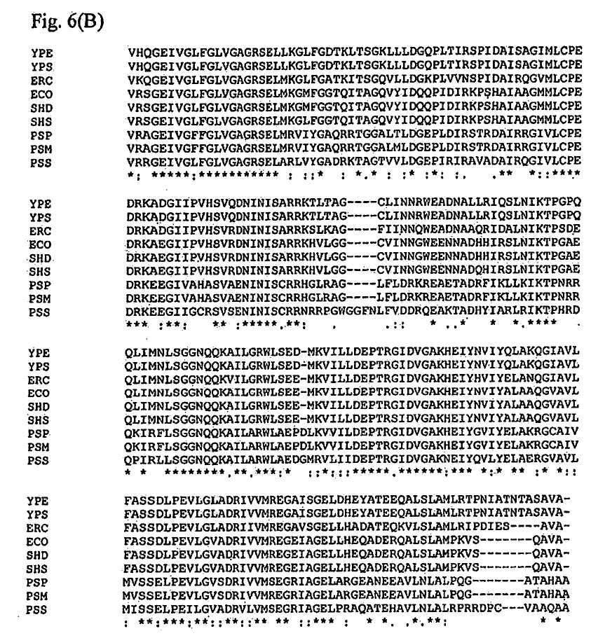

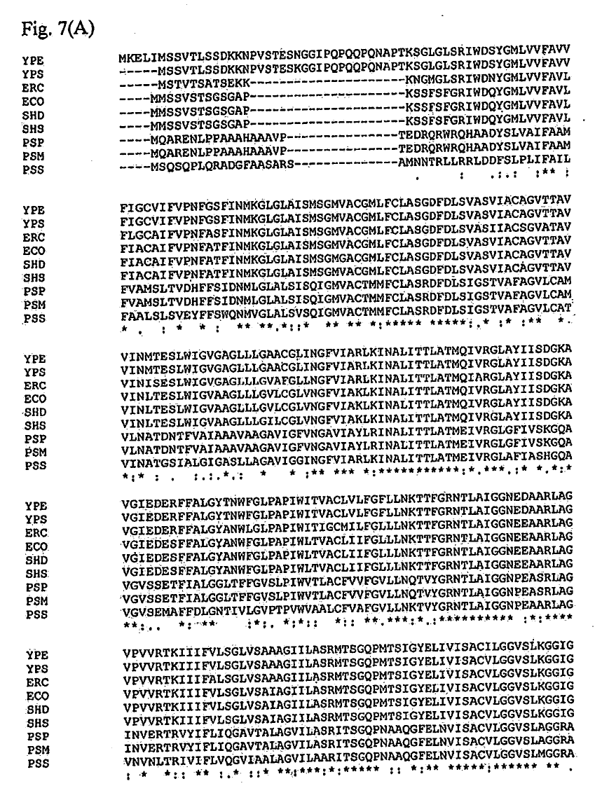

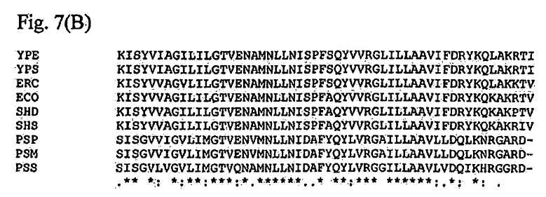

Data comparing the primary sequences of ara FGH from Escherichia coli (ECO), Shigella dysenteriae serotype I (SHD), Shigella sonney (SHS), Erwinia carotovora subsp. atroseptica (ERC), Yersinia pestis (YPE), Yersinia pseudotuberculosis (YPS), Pseudomonas pseudomallei (PSP), Pseudomonas mallei (PSM), Pseudomonas solanacearum (PSS) show a high level of homology among these proteins (see FIG. 5, FIG. 6, FIG. 7). From this point of view, substitutions or deletions of the amino acid residues which are identical (marked by asterisk) in all the above-mentioned proteins are likely crucial for their function. It is possible to substitute similar (marked by colon) amino acids residues by the similar amino acid residues without deterioration of the protein activity. But modifications of other non-conserved amino acid residues may not lead to alteration of the activity of high-affinity L-arabinose transporter.

The DNAs which encode substantially the same proteins as components of L-arabinose transporter may be obtained, for example, by modifying the nucleotide sequences of DNAs encoding components of L-arabinose transporter (SEQ ID NO: 1, SEQ ID NO: 3 or SEQ ID NO: 5 respectively), for example, by means of the site-directed mutagenesis method so that one or more amino acid residues at a specified site are deleted, substituted, inserted, or added.

DNAs modified as described above may be obtained by conventionally known mutation treatments. Such treatments include hydroxylamine treatment of the DNA encoding proteins of present invention, or treatment of the bacterium containing the DNA with UV irradiation or a reagent such as N-methyl-N′-nitro-N-nitrosoguanidine or nitrous acid. DNAs encoding substantially the same proteins as components of L-arabinose transporter can be obtained by expressing DNAs having a mutation as described above in an appropriate cell, and investigating the activity of the expressed product. DNAs encoding substantially the same protein as components of L-arabinose transporter can also be obtained by isolating DNAs that are hybridizable with probes having nucleotide sequences which contain, for example, the nucleotide sequences shown in any of SEQ ID NO: 1, SEQ ID NO: 3, and SEQ ID NO: 5 under the stringent conditions, and encode proteins having the activities of components of L-arabinose transporter. The “stringent conditions” referred to herein are conditions under which so-called specific hybrids are formed, and non-specific hybrids are not formed. For example, stringent conditions can be exemplified by conditions under which DNAs having high homology, for example, DNAs having homology of not less than 50%, preferably not less than 60%, more preferably not less than 70%, further preferably not less than 80%, and still more preferably not less than 90%, and most preferably not less than 95% are able to hybridize with each other, but DNAs having homology lower than the above are not able to hybridize with each other. Alternatively, stringent conditions may be exemplified by conditions under which DNA is able to hybridize at a salt concentration equivalent to ordinary washing conditions in Southern hybridization, i.e., 1×SSC, 0.1% SDS, preferably 0.1×SSC, 0.1% SDS, at 60° C. Duration of washing depends on the type of membrane used for blotting and, as a rule, what is recommended by the manufacturer. For example, recommended duration of washing, for example, for the Hybond™ N+ nylon membrane (Amersham), under stringent conditions is approximately 15 minutes. Preferably, washing is performed 2 to 3 times.

Partial sequences of the nucleotide sequences of SEQ ID NO: 1, SEQ ID NO: 3, SEQ ID NO: 5 can also be used as probes. Probes may be prepared by PCR using primers based on the nucleotide sequences of SEQ ID NO: 1, SEQ ID NO: 3, SEQ ID NO: 5 and DNA fragments containing the nucleotide sequences of SEQ ID NO: 1, SEQ ID NO: 3, SEQ ID NO: 5 as templates. When a DNA fragment having a length of about 300 bp is used as the probe, the hybridization conditions for washing include, for example, 50° C., 2×SSC and 0.1% SDS.

The substitution, deletion, insertion, or addition of nucleotides as described above also may include a mutation which naturally occurs (mutant or variant), for example, due to variety in the species or genus of bacterium which contains the components of the L-arabinose transporter.

“Transformation of a bacterium with DNA encoding a protein” means introduction of the DNA into a bacterium, for example, by conventional methods. Transformation of this DNA will result in an increase in expression of the gene encoding the protein of the present invention, and will enhance the activity of the protein in the bacterial cell. Methods of transformation include any known methods that have hitherto been reported. For example, a method of treating recipient cells with calcium chloride so as to increase permeability of the cells to DNA has been reported for Escherichia coli K-12 (Mandel, M. and Higa, A., J. Mol. Biol., 53, 159 (1970)) and may be used.

Methods of enhancing gene expression include increasing the gene copy number. Introducing a gene into a vector that is able to function in a bacterium of the Enterobacteriaceae family increases the copy number of the gene. Preferably, low copy vectors are used. Examples of low-copy vectors include but are not limited to pSC101, pMW118, pMW119, and the like. The term “low copy vector” applies to vectors which have up to 5 copies per cell.

Increasing the copy number of the araFGH operon can also be achieved by introducing multiple copies of the araFGH operon into the chromosomal DNA of the bacterium. In order to introduce multiple copies of the operon into a bacterial chromosome, homologous recombination is carried out using a sequence whose multiple copies exist as targets in the chromosomal DNA. Sequences having multiple copies in the chromosomal DNA include, but are not limited to repetitive DNA, or inverted repeats existing at the end of a transposable element. Also, as disclosed in U.S. Pat. No. 5,595,889, it is possible to incorporate the araFGH operon into a transposon, and allow it to be transferred to introduce multiple copies of the gene into the chromosomal DNA. Introduction of multiple copies of the gene into a bacterial chromosome can be also achieved by Mu integration, or the like. For example, one act of Mu integration allows introduction of up to 3 copies of the gene into a bacterial chromosome.

Enhancing gene expression may also be achieved by placing the DNA under the control of a potent promoter. For example, the Ptac promoter, the ac promoter, the trp promoter, the trc promoter, the PR, or the PL promoters of lambda phage are all known to be potent promoters. The use of a potent promoter can be combined with increasing the gene copy number.

Alternatively, the effect of a promoter can be enhanced by, for example, introducing a mutation into the promoter to increase the transcription level of a gene located downstream of the promoter. Furthermore, it is known that substitution of several nucleotides in the spacer between ribosome binding site (RBS) and the start codon, especially the sequences immediately upstream of the start codon, profoundly affect the mRNA translatability. For example, a 20-fold range in the expression levels was found, depending on the nature of the three nucleotides preceding the start codon (Gold et al., Annu. Rev. Microbiol., 35, 365-403, 1981; Hui et al., EMBO J., 3, 623-629, 1984). Previously, it was shown that the rhtA23 mutation is an A-for-G substitution at the −1 position relative to the ATG start codon (ABSTRACTS of 17^{th} International Congress of Biochemistry and Molecular Biology in conjugation with 1997 Annual Meeting of the American Society for Biochemistry and Molecular Biology, San Francisco, Calif. Aug. 24-29, 1997, abstract No. 457). Therefore, it may be suggested that the rhtA23 mutation enhances the rhtA gene expression and, as a consequence, increases the resistance to threonine, homoserine, and some other substances transported out of cells.

Moreover, it is also possible to introduce a nucleotide substitution into the promoter region of the araFGH operon on the bacterial chromosome, which results in stronger promoter function. The alteration of the expression control sequence can be performed, for example, in the same manner as the gene substitution using a temperature-sensitive plasmid, as disclosed in International Patent Publication WO 00/18935 and Japanese Patent Application Laid-Open No. JP 1-215280 A.

Methods for preparation of plasmid DNA include, but are not limited to digestion and ligation of DNA, transformation, selection of an oligonucleotide as a primer and the like, or other methods well known to one skilled in the art. These methods are described, for instance, in Sambrook, J., Fritsch, E. F., and Maniatis, T., “Molecular Cloning A Laboratory Manual, Second Edition”, Cold Spring Harbor Laboratory Press (1989).

The above-described techniques and guidances for enhancing an activity of arabinose transporter are similarly applied to enhancing activities of xylose isomerase and glucokinase. The bacterium of the present invention can be obtained by the introduction of the aforementioned DNAs into a bacterium which inherently has the ability to produce L-amino acids. Alternatively, the bacterium of the present invention can be obtained by imparting an ability to produce L-amino acids to a bacterium which already contains the DNAs.

L-Amino Acid-Producing Bacteria

As a bacterium which is modified to enhance expression of the araFGH genes, bacteria which are able to produce L-amino acids may be used.

The bacterium can be obtained by enhancing expression of the araFGH genes in a bacterium which inherently has the ability to produce L-amino acids. Alternatively, the bacterium can be obtained by imparting the ability to produce L-amino acids to a bacterium already having the enhanced expression of the araFGH genes.

L-Threonine-Producing Bacteria

Examples of parent strains for deriving L-threonine-producing bacteria include, but are not limited to, strains belonging to the genus Escherichia, such as E. coli TDH-6/pVIC40 (VKPM B-3996) (U.S. Pat. No. 5,175,107, U.S. Pat. No. 5,705,371), E. coli 472T23/pYN7 (ATCC 98081) (U.S. Pat. No. 5,631,157), E. coli NRRL-21593 (U.S. Pat. No. 5,939,307), E. coli FERM BP-3756 (U.S. Pat. No. 5,474,918), E. coli FERM BP-3519 and FERM BP-3520 (U.S. Pat. No. 5,376,538), E. coli MG442 (Gusyatiner et al., Genetika (in Russian), 14, 947-956 (1978)), E. coli VL643 and VL2055 (EP 1149911 A), and the like.

The strain TDH-6 is deficient in the thrC gene, as well as being sucrose-assimilative, and the ilvA gene has a leaky mutation. This strain also has a mutation in the rhtA gene, which imparts resistance to high concentrations of threonine or homoserine. The strain B-3996 contains the plasmid pVIC40 which was obtained by inserting a thrA*BC operon which includes a mutant thrA gene into a RSF110-derived vector. This mutant thrA gene encodes aspartokinase homoserine dehydrogenase I which has substantially desensitized feedback inhibition by threonine. The strain B-3996 was deposited on Nov. 19, 1987 in the All-Union Scientific Center of Antibiotics (Russia, 117105 Moscow, Nagatinskaya Street 3-A) under the accession number RIA 1867. The strain was also deposited in the Russian National Collection of Industrial Microorganisms (VKPM) (Russia, 117545 Moscow, 1 Dorozhny proezd, 1) on Apr. 7, 1987 under the accession number VKPM B-3996.

E. coli VKPM B-5318 (EP0593792B) may also be used as a parent strain for deriving L-threonine-producing bacteria. The strain B-5318 is prototrophic with regard to isoleucine, and a temperature-sensitive lambda-phage C1 repressor and PR promoter replaces the regulatory region of the threonine operon in plasmid pVIC40. The strain VKPM B-5318 was deposited in the Russian National Collection of Industrial Microorganisms (VKPM) on May 3, 1990 under accession number of VKPM B-5318.

Preferably, the bacterium is additionally modified to enhance expression of one or more of the following genes:
the mutant thrA gene which codes for aspartokinase homoserine dehydrogenase I resistant to feed back inhibition by threonine;
the thrB gene which codes for homoserine kinase;
the thrC gene which codes for threonine synthase;
the rhtA gene which codes for a putative transmembrane protein;
the asd gene which codes for aspartate-α-semialdehyde dehydrogenase; and
the aspC gene which codes for aspartate aminotransferase (aspartate transaminase);

The thrA gene which encodes aspartokinase homoserine dehydrogenase I of Escherichia coli has been elucidated (nucleotide positions 337 to 2799, GenBank accession NC_{—}000913.2, gi: 49175990). The thrA gene is located between the thrL and thrB genes on the chromosome of E. coli K-12. The thrB gene which encodes homoserine kinase of Escherichia coli has been elucidated (nucleotide positions 2801 to 3733, GenBank accession NC_{—}000913.2, gi: 49175990). The thrB gene is located between the thrA and thrC genes on the chromosome of E. coli K-12. The thrC gene which encodes threonine synthase of Escherichia coli has been elucidated (nucleotide positions 3734 to 5020, GenBank accession NC_{—}000913.2, gi: 49175990). The thrC gene is located between the thrB gene and the yaaX open reading frame on the chromosome of E. coli K-12. All three genes function as a single threonine operon. To enhance expression of the threonine operon, the attenuator region which affects the transcription is desirably removed from the operon (WO2005/049808, WO2003/097839).

A mutant thrA gene which codes for aspartokinase homoserine dehydrogenase I resistant to feed back inhibition by threonine, as well as, the thrB and thrC genes can be obtained as one operon from the well-known plasmid pVIC40 which is present in the threonine producing E. coli strain VKPM B-3996. Plasmid pVIC40 is described in detail in U.S. Pat. No. 5,705,371.

The rhtA gene exists at 18 min on the E. coli chromosome close to the glnHPQ operon, which encodes components of the glutamine transport system. The rhtA gene is identical to ORF1 (ybiF gene, nucleotide positions 764 to 1651, GenBank accession number AAA218541, gi:440181) and is located between the pexB and ompX genes. The unit expressing a protein encoded by the ORF1 has been designated the rhtA gene (rht: resistance to homoserine and threonine). Also, it was revealed that the rhtA23 mutation is an A-for-G substitution at position −1 with respect to the ATG start codon (ABSTRACTS of the 17^{th} International Congress of Biochemistry and Molecular Biology in conjugation with Annual Meeting of the American Society for Biochemistry and Molecular Biology, San Francisco, Calif. Aug. 24-29, 1997, abstract No. 457, EP 1013765 A).

The asd gene of E. coli has already been elucidated (nucleotide positions 3572511 to 3571408, GenBank accession NC_{—}000913.1, gi:16131307), and can be obtained by PCR (polymerase chain reaction; refer to White, T. J. et al., Trends Genet., 5, 185 (1989)) utilizing primers prepared based on the nucleotide sequence of the gene. The asd genes of other microorganisms can be obtained in a similar manner.

Also, the aspC gene of E. coli has already been elucidated (nucleotide positions 983742 to 984932, GenBank accession NC_{—}000913.1, gi:16128895), and can be obtained by PCR. The aspC genes of other microorganisms can be obtained in a similar manner.

L-Lysine-Producing Bacteria

Examples of L-lysine-producing bacteria belonging to the genus Escherichia include mutants having resistance to an L-lysine analogue. The L-lysine analogue inhibits growth of bacteria belonging to the genus Escherichia, but this inhibition is fully or partially desensitized when L-lysine coexists in a medium. Examples of the L-lysine analogue include, but are not limited to, oxalysine, lysine hydroxamate, S-(2-aminoethyl)-L-cysteine (AEC), γ-methyllysine, α-chlorocaprolactam and so forth. Mutants having resistance to these lysine analogues can be obtained by subjecting bacteria belonging to the genus Escherichia to a conventional artificial mutagenesis treatment. Specific examples of bacterial strains useful for producing L-lysine include Escherichia coli AJ11442 (FERM BP-1543, NRRL B-12185; see U.S. Pat. No. 4,346,170) and Escherichia coli VL611. In these microorganisms, feedback inhibition of aspartokinase by L-lysine is desensitized.

The strain WC196 may be used as an L-lysine producing bacterium of Escherichia coli. This bacterial strain was bred by conferring AEC resistance to the strain W3110, which was derived from Escherichia coli K-12. The resulting strain was designated Escherichia coli AJ13069 and was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on Dec. 6, 1994 and received an accession number of FERM P-14690. Then, it was converted to an international deposit under the provisions of the Budapest Treaty on Sep. 29, 1995, and received an accession number of FERM BP-5252 (U.S. Pat. No. 5,827,698).

Examples of parent strains for deriving L-lysine-producing bacteria also include strains in which expression of one or more genes encoding an L-lysine biosynthetic enzyme are enhanced. Examples of such genes include, but are not limited to, genes encoding dihydrodipicolinate synthase (dapA), aspartokinase (lysC), dihydrodipicolinate reductase (dapB), diaminopimelate decarboxylase (lysA), diaminopimelate dehydrogenase (ddh) (U.S. Pat. No. 6,040,160), phosphoenolpyrvate carboxylase (ppc), aspartate semialdehyde dehydrogenease (asd), and aspartase (aspA) (EP 1253195 A). In addition, the parent strains may have an increased level of expression of the gene involved in energy efficiency (cyo) (EP 1170376 A), the gene encoding nicotinamide nucleotide transhydrogenase (pntAB) (U.S. Pat. No. 5,830,716), the ybjE gene (WO2005/073390), or combinations thereof.

Examples of parent strains for deriving L-lysine-producing bacteria also include strains having decreased or eliminated activity of an enzyme that catalyzes a reaction for generating a compound other than L-lysine by branching off from the biosynthetic pathway of L-lysine. Examples of the enzymes that catalyze a reaction for generating a compound other than L-lysine by branching off from the biosynthetic pathway of L-lysine include homoserine dehydrogenase, lysine decarboxylase (U.S. Pat. No. 5,827,698), and the malic enzyme (WO2005/010175).

L-Cysteine-Producing Bacteria

Examples of parent strains for deriving L-cysteine-producing bacteria include, but are not limited to, strains belonging to the genus Escherichia, such as E. coli JM15 which is transformed with different cysE alleles coding for feedback-resistant serine acetyltransferases (U.S. Pat. No. 6,218,168, Russian patent application 2003121601); E. coli W3110 having over-expressed genes which encode proteins suitable for secreting substances toxic for cells (U.S. Pat. No. 5,972,663); E. coli strains having lowered cysteine desulfohydrase activity (JP11155571A2); E. coli W3110 with increased activity of a positive transcriptional regulator for cysteine regulon encoded by the cysB gene (WO0127307A1), and the like.

L-Leucine-Producing Bacteria

Examples of parent strains for deriving L-leucine-producing bacteria include, but are not limited to, strains belonging to the genus Escherichia, such as E. coli strains resistant to leucine (for example, the strain 57 (VKPM B-7386, U.S. Pat. No. 6,124,121)) or leucine analogs including β-2-thienylalanine, 3-hydroxyleucine, 4-azaleucine, 5,5,5-trifluoroleucine (JP 62-34397 B and JP 8-70879 A); E. coli strains obtained by the gene engineering method described in WO96/06926; E. coli H-9068 (JP 8-70879 A), and the like.

The bacterium may be improved by enhancing the expression of one or more genes involved in L-leucine biosynthesis. Examples include genes of the leuABCD operon, which are preferably represented by a mutant leuA gene coding for isopropylmalate synthase freed from feedback inhibition by L-leucine (U.S. Pat. No. 6,403,342). In addition, the bacterium may be improved by enhancing the expression of one or more genes coding for proteins which excrete L-amino acid from the bacterial cell. Examples of such genes include the b2682 and b2683 genes (ygaZH genes) (EP 1239041 A2).

L-Histidine-Producing Bacteria

Examples of parent strains for deriving L-histidine-producing bacteria include, but are not limited to, strains belonging to the genus Escherichia, such as E. coli strain 24 (VKPM B-5945, RU2003677); E. coli strain 80 (VKPM B-7270, RU2119536); E. coli NRRL B-12116-B12121 (U.S. Pat. No. 4,388,405); E. coli H-9342 (FERM BP-6675) and H-9343 (FERM BP-6676) (U.S. Pat. No. 6,344,347); E. coli H-9341 (FERM BP-6674) (EP1085087); E. coli A180/pFM201 (U.S. Pat. No. 6,258,554) and the like.

Examples of parent strains for deriving L-histidine-producing bacteria also include strains in which expression of one or more genes encoding an L-histidine biosynthetic enzyme are enhanced. Examples of such genes include genes encoding ATP phosphoribosyltransferase (hisG), phosphoribosyl AMP cyclohydrolase (hisI), phosphoribosyl-ATP pyrophosphohydrolase (hisIE), phosphoribosylformimino-5-aminoimidazole carboxamide ribotide isomerase (hisA), amidotransferase (hisH), histidinol phosphate aminotransferase (hisC), histidinol phosphatase (hisB), histidinol dehydrogenase (hisD), and so forth.

It is known that the L-histidine biosynthetic enzymes encoded by hisG and hisBHAFI are inhibited by L-histidine, and therefore an L-histidine-producing ability can also be efficiently enhanced by introducing a mutation which confers resistance to the feedback inhibition into ATP phosphoribosyltransferase (Russian Patent Nos. 2003677 and 2119536).

Specific examples of strains having an L-histidine-producing ability include E. coli FERM-P 5038 and 5048 which have been introduced with a vector carrying a DNA encoding an L-histidine-biosynthetic enzyme (JP 56-005099 A), E. coli strains introduced with rht, a gene for an amino acid-export (EP1016710A), E. coli 80 strain imparted with sulfaguanidine, DL-1,2,4-triazole-3-alanine, and streptomycin-resistance (VKPM B-7270, Russian Patent No. 2119536), and so forth.

L-Glutamic Acid-Producing Bacteria

Examples of parent strains for deriving L-glutamic acid-producing bacteria include, but are not limited to, strains belonging to the genus Escherichia, such as E. coli VL334thrC⁺ (EP 1172433). E. coli VL334 (VKPM B-1641) is an L-isoleucine and L-threonine auxotrophic strain having mutations in thrC and ilvA genes (U.S. Pat. No. 4,278,765). A wild-type allele of the thrC gene was transferred by the method of general transduction using a bacteriophage P1 grown on the wild-type E. coli strain K12 (VKPM B-7) cells. As a result, an L-isoleucine auxotrophic strain VL334thrC⁺ (VKPM B-8961), which is able to produce L-glutamic acid, was obtained.

Examples of parent strains for deriving the L-glutamic acid-producing bacteria include, but are not limited to, strains in which expression of one or more genes encoding an L-glutamic acid biosynthetic enzyme are enhanced. Examples of such genes include genes encoding glutamate dehydrogenase (gdhA), glutamine synthetase (glnA), glutamate synthetase (gltAB), isocitrate dehydrogenase (icdA), aconitate hydratase (acnA, acnB), citrate synthase (gltA), phosphoenolpyruvate carboxylase (ppc), pyruvate carboxylase (pyc), pyruvate dehydrogenase (aceEF, lpdA), pyruvate kinase (pyka, pykF), phosphoenolpyruvate synthase (ppsA), enolase (eno), phosphoglyceromutase (pgmA, pgmI), phosphoglycerate kinase (pgk), glyceraldehyde-3-phophate dehydrogenase (gapA), triose phosphate isomerase (tpiA), fructose bisphosphate aldolase (fbp), phosphofructokinase (pfkA, pfkB), and glucose phosphate isomerase (pgi).

Examples of strains modified so that expression of the citrate synthetase gene, the phosphoenolpyruvate carboxylase gene, and/or the glutamate dehydrogenase gene is/are enhanced include those disclosed in EP1078989A, EP955368A, and EP952221A.

Examples of parent strains for deriving the L-glutamic acid-producing bacteria also include strains having decreased or eliminated activity of an enzyme that catalyzes synthesis of a compound other than L-glutamic acid by branching off from an L-glutamic acid biosynthesis pathway. Examples of such enzymes include isocitrate lyase (aceA), α-ketoglutarate dehydrogenase (sucA), phosphotransacetylase (pta), acetate kinase (ack), acetohydroxy acid synthase (ilvG), acetolactate synthase (ilvI), formate acetyltransferase (pfl), lactate dehydrogenase (ldh), and glutamate decarboxylase (gadAB). Bacteria belonging to the genus Escherichia deficient in α-ketoglutarate dehydrogenase activity or having reduced α-ketoglutarate dehydrogenase activity and methods for obtaining them are described in U.S. Pat. Nos. 5,378,616 and 5,573,945. Specifically, these strains include the following:

E. coli W3110sucA::Km^{R}

E. coli AJ12624 (FERM BP-3853)

E. coli AJ12628 (FERM BP-3854)

E. coli AJ12949 (FERM BP-4881)

E. coli W3110sucA::Km^{R} is a strain obtained by disrupting the α-ketoglutarate dehydrogenase gene (hereinafter referred to as “sucA gene”) of E. coli W3110. This strain is completely deficient in α-ketoglutarate dehydrogenase.

Other examples of L-glutamic acid-producing bacterium include those which belong to the genus Escherichia and have resistance to an aspartic acid antimetabolite. These strains can also be deficient in α-ketoglutarate dehydrogenase activity and include, for example, E. coli AJ13199 (FERM BP-5807) (U.S. Pat. No. 5,908,768), FFRM P-12379, which additionally has a low L-glutamic acid decomposing ability (U.S. Pat. No. 5,393,671); AJ13138 (FERM BP-5565) (U.S. Pat. No. 6,110,714), and the like.

Examples of L-glutamic acid-producing bacteria include mutant strains belonging to the genus Pantoea which are deficient in α-ketoglutarate dehydrogenase activity or have a decreased α-ketoglutarate dehydrogenase activity, and can be obtained as described above. Such strains include Pantoea ananatis AJ13356. (U.S. Pat. No. 6,331,419). Pantoea ananatis AJ13356 was deposited at the National Institute of Bioscience and Human-Technology, Agency of Industrial Science and Technology, Ministry of International Trade and Industry (currently, National Institute of Advanced Industrial Science and Technology, International Patent Organism Depositary, Central 6, 1-1, Higashi 1-Chome, Tsukuba-shi, Ibaraki-ken, 305-8566, Japan) on Feb. 19, 1998 under an accession number of FERM P-16645. It was then converted to an international deposit under the provisions of Budapest Treaty on Jan. 11, 1999 and received an accession number of FERM BP-6615. Pantoea ananatis AJ13356 is deficient in α-ketoglutarate dehydrogenase activity as a result of disruption of the αKGDH-E1 subunit gene (sucA). The above strain was identified as Enterobacter agglomerans when it was isolated and deposited as the Enterobacter agglomerans AJ13356. However, it was recently re-classified as Pantoea ananatis on the basis of nucleotide sequencing of 16S rRNA and so forth. Although AJ13356 was deposited at the aforementioned depository as Enterobacter agglomerans, for the purposes of this specification, they are described as Pantoea ananatis.

L-Phenylalanine-Producing Bacteria

Examples of parent strains for deriving L-phenylalanine-producing bacteria include, but are not limited to, strains belonging to the genus Escherichia, such as E. coli AJ12739 (tyrA::Tn10, tyrR) (VKPM B-8197); E. coli HW1089 (ATCC 55371) harboring the mutant pheA34 gene (U.S. Pat. No. 5,354,672); E. coli MWEC101-b (KR8903681); E. coli NRRL B-12141, NRRL B-12145, NRRL B-12146 and NRRL B-12147 (U.S. Pat. No. 4,407,952). Also, as a parent strain, E. coli K-12 [W3110 (tyrA)/pPHAB (FERM BP-3566), E. coli K-12 [W3110 (tyrA)/pPHAD] (FERM BP-12659), E. coli K-12 [W3110 (tyrA)/pPHATerm] (FERM BP-12662) and E. coli K-12 [W3110 (tyrA)/pBR-aroG4, pACMAB] named as AJ 12604 (FERM BP-3579) may be used (EP 488-424 B1). Furthermore, L-phenylalanine producing bacteria belonging to the genus Escherichia with an enhanced activity of the protein encoded by the yedA gene or the yddG gene may also be used (U.S. patent applications 2003/0148473 A1 and 2003/0157667 A1).

L-Tryptophan-Producing Bacteria

Examples of parent strains for deriving the L-tryptophan-producing bacteria include, but are not limited to, strains belonging to the genus Escherichia, such as E. coli JP4735/pMU3028 (DSM10122) and JP6015/pMU91 (DSM10123) are deficient in the tryptophanyl-tRNA synthetase encoded by mutant trpS gene (U.S. Pat. No. 5,756,345); E. coli SV164 (pGH5) having a serA allele encoding phosphoglycerate dehydrogenase free from feedback inhibition by serine and a trpE allele encoding anthranilate synthase free from feedback inhibition by tryptophan (U.S. Pat. No. 6,180,373); E. coli AGX17 (pGX44) (NRRL B-12263) and AGX6(pGX50)aroP (NRRL B-12264) deficient in the enzyme tryptophanase (U.S. Pat. No. 4,371,614); E. coli AGX17/pGX50, pACKG4-pps in which a phosphoenolpyruvate-producing ability is enhanced (WO9708333, U.S. Pat. No. 6,319,696), and the like may be used. L-tryptophan-producing bacteria belonging to the genus Escherichia with an enhanced activity of the identified protein encoded by and the yedA gene or the yddG gene may also be used (U.S. patent applications 2003/0148473 A1 and 2003/0157667 A1).

Examples of parent strains for deriving the L-tryptophan-producing bacteria also include strains in which one or more activities of the enzymes selected from anthranilate synthase, phosphoglycerate dehydrogenase, and tryptophan synthase are enhanced. The anthranilate synthase and phosphoglycerate dehydrogenase are both subject to feedback inhibition by L-tryptophan and L-serine, so that a mutation desensitizing the feedback inhibition may be introduced into these enzymes. Specific examples of strains having such a mutation include a E. coli SV164 which harbors desensitized anthranilate synthase and a transformant strain obtained by introducing into the E. coli SV164 the plasmid pGH5 (WO 94/08031), which contains a mutant serA gene encoding feedback-desensitized phosphoglycerate dehydrogenase.

Examples of parent strains for deriving the L-tryptophan-producing bacteria also include strains into which the tryptophan operon which contains a gene encoding desensitized anthranilate synthase has been introduced (JP 57-71397 A, JP 62-244382 A, U.S. Pat. No. 4,371,614). Moreover, L-tryptophan-producing ability may be imparted by enhancing expression of a gene which encodes tryptophan synthase, among tryptophan operons (trpBA). The tryptophan synthase consists of α and β subunits which are encoded by the trpA and trpB genes, respectively. In addition, L-tryptophan-producing ability may be improved by enhancing expression of the isocitrate lyase-malate synthase operon (WO2005/103275).

L-Proline-Producing Bacteria

Examples of parent strains for deriving L-proline-producing bacteria include, but are not limited to, strains belonging to the genus Escherichia, such as E. coli 702ilvA (VKPM B-8012) which is deficient in the ilvA gene and is able to produce L-proline (EP 1172433). The bacterium may be improved by enhancing the expression of one or more genes involved in L-proline biosynthesis. Examples of such genes for L-proline producing bacteria which are preferred include the proB gene coding for glutamate kinase of which feedback inhibition by L-proline is desensitized (DE Patent 3127361). In addition, the bacterium may be improved by enhancing the expression of one or more genes coding for proteins excreting L-amino acid from bacterial cell. Such genes are exemplified by b2682 and b2683 genes (ygaZH genes) (EP1239041 A2).

Examples of bacteria belonging to the genus Escherichia, which have an activity to produce L-proline include the following E. coli strains: NRRL B-12403 and NRRL B-12404 (GB Patent 2075056), VKPM B-8012 (Russian patent application 2000124295), plasmid mutants described in DE Patent 3127361, plasmid mutants described by Bloom F. R. et al (The 15^{th} Miami winter symposium, 1983, p. 34), and the like.

L-Arginine-Producing Bacteria

Examples of parent strains for deriving L-arginine-producing bacteria include, but are not limited to, strains belonging to the genus Escherichia, such as E. coli strain 237 (VKPM B-7925) (U.S. Patent Application 2002/058315 A1) and its derivative strains harboring mutant N-acetylglutamate synthase (Russian Patent Application No. 2001112869), E. coli strain 382 (VKPM B-7926) (EP1170358A1), an arginine-producing strain into which argA gene encoding N-acetylglutamate synthetase is introduced therein (EP1170361A1), and the like.

Examples of parent strains for deriving L-arginine producing bacteria also include strains in which expression of one or more genes encoding an L-arginine biosynthetic enzyme are enhanced. Examples of such genes include genes encoding N-acetylglutamyl phosphate reductase (argC), ornithine acetyl transferase (argJ), N-acetylglutamate kinase (argB), acetylornithine transaminase (argD), ornithine carbamoyl transferase (argF), argininosuccinic acid synthetase (argG), argininosuccinic acid lyase (argH), and carbamoyl phosphate synthetase (carAB).

L-Valine-Producing Bacteria

Example of parent strains for deriving L-valine-producing bacteria include, but are not limited to, strains which have been modified to overexpress the ilvGMEDA operon (U.S. Pat. No. 5,998,178). It is desirable to remove the region of the ilvGMEDA operon which is required for attenuation so that expression of the operon is not attenuated by the L-valine that is produced. Furthermore, the ilvA gene in the operon is desirably disrupted so that threonine deaminase activity is decreased.

Examples of parent strains for deriving L-valine-producing bacteria include also include mutants having a mutation of amino-acyl t-RNA synthetase (U.S. Pat. No. 5,658,766). For example, E. coli VL1970, which has a mutation in the ileS gene encoding isoleucine tRNA synthetase, can be used. E. coli VL1970 has been deposited in the Russian National Collection of Industrial Microorganisms (VKPM) (Russia, 117545 Moscow, 1 Dorozhny Proezd, 1) on Jun. 24, 1988 under accession number VKPM B-4411.

Furthermore, mutants requiring lipoic acid for growth and/or lacking H⁺-ATPase can also be used as parent strains (WO96/06926).

L-Isoleucine-Producing Bacteria

Examples of parent strains for deriving L-isoleucine producing bacteria include, but are not limited to, mutants having resistance to 6-dimethylaminopurine (JP 5-304969 A), mutants having resistance to an isoleucine analogue such as thiaisoleucine and isoleucine hydroxamate, and mutants additionally having resistance to DL-ethionine and/or arginine hydroxamate (JP 5-130882 A). In addition, recombinant strains transformed with genes encoding proteins involved in L-isoleucine biosynthesis, such as threonine deaminase and acetohydroxate synthase, can also be used as parent strains (JP 2-458 A, FR 0356739, and U.S. Pat. No. 5,998,178).

2. Method

Oxaloacetate (OAA) serves as a substrate for the reaction which results in the synthesis of Thr and Lys. OAA results from a reaction of PEP with phosphoenol pyrvate carboxlase (PEPC) functioning as a catalyst. Therefore, elevation of the PEPC concentration in a cell can be very important for fermentative production of these amino acids. When using glucose as the carbon source in fermentation, glucose is internalized by the glucose-phosphontransferase (Glc-PTS) system. This system consumes PEP, and proteins in the PTS are encoded by ptsG and ptsHIcrr. During internalization, one molecule of PEP and one molecule of pyruvate (Pyr) are generated from one molecule of glucose.

An L-threonine-producing strain and an L-lysine-producing strain which have been modified to have an ability to utilize sucrose (Scr-PTS) have higher productivity of these amino acids when cultured in sucrose rather than glucose (EP 1149911 A2). It is believed that three molecules of PEP and one molecule of Pyr are generated from one molecule of sucrose by the Scr-PTS, increasing the ratio of PEP/Pyr, and thereby facilitating the synthesis of Thr and Lys from sucrose. Furthermore, it has been reported that Glc-PTS is subject to several expression controls (Postma P. W. et al., Microbiol Rev., 57(3), 543-94 (1993); Clark B. et al. J. Gen. Microbiol., 96(2), 191-201 (1976); Plumbridge J., Curr. Opin. Microbiol., 5(2), 187-93 (2002); Ryu S. et al., J. Biol. Chem., 270(6):2489-96 (1995)), and hence it is possible that the incorporation of glucose itself can be a rate-limiting step in amino acid fermentation.

Increasing the ratio of PEP/Pyr even more by increasing expression of the araFGH operon in a threonine-producing strain, a lysine-producing strain, a histidine-producing strain, a phenylalanine-producing strain, an arginine-producing strain, a tryptophan-producing strain and/or a glutamic acid-producing strain should further increase the corresponding amino acid production. Because four molecules of PEP are generated from two molecules of glucose, the ratio of PEP/Pyr is expected to be greatly improved. Due to the increased expression of the araFGH operon, removal of the expression control glc-PTS is expected.

The method of the present invention is a method for producing an L-amino acid by cultivating the bacterium in a culture medium to produce and excrete the L-amino acid into the medium, and collecting the L-amino acid from the medium.

The cultivation, collection, and purification of an L-amino acid from the medium and the like may be performed in a manner similar to conventional fermentation methods wherein an amino acid is produced using a bacterium.

A medium used for culture may be either a synthetic or natural medium, so long as the medium includes a carbon source and a nitrogen source and minerals and, if necessary, appropriate amounts of nutrients which the bacterium requires for growth. The carbon source may include various carbohydrates such as glucose and sucrose, and various organic acids. Depending on the mode of assimilation of the chosen microorganism, alcohol, including ethanol and glycerol, may be used. As the nitrogen source, various ammonium salts such as ammonia and ammonium sulfate, other nitrogen compounds such as amines, a natural nitrogen source such as peptone, soybean-hydrolysate, and digested fermentative microorganism can be used. As minerals, potassium monophosphate, magnesium sulfate, sodium chloride, ferrous sulfate, manganese sulfate, calcium chloride, and the like can be used. As vitamins, thiamine, yeast extract, and the like, can be used.

The cultivation is preferably performed under aerobic conditions, such as a shaking culture, and a stirring culture with aeration, at a temperature of 20 to 40° C., preferably 30 to 38° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to accumulation of the target L-amino acid in the liquid medium.

After cultivation, solids such as cells can be removed from the liquid medium by centrifugation or membrane filtration, and then the L-amino acid can be collected and purified by ion-exchange, concentration, and/or crystallization methods.

### EXAMPLES

The present invention will be more concretely explained below with reference to the following non-limiting Examples.

### Example 1

### Construction of the E. coli Strain Having a Disrupted PTS Transport System

1. Deletion of the ptsHI-crr Operon

The ptsHI-crr operon was deleted in a chosen strain by the method initially developed by Datsenko, K. A. and Wanner, B. L. (Proc. Natl. Acad. Sci. USA, 2000, 97(12): 6640-6645) called “Red-driven integration”. The DNA fragment containing the Cm^{R} marker encoded by the cat gene was obtained by PCR, using primers P1 (SEQ ID NO: 7) and P2 (SEQ ID NO: 8) and plasmid pMW118-attL-Cm-attR as a template (WO 05/010175). Primer P1 contains both a region complementary to the 36-nt region located at the 5′ end of the ptsHI-crr operon, and a region complementary to the 24-nt attL region. Primer P2 contains both a region complementary to the 36-nt region located at the 3′ end of the ptsHI-crr operon, and a region complementary to the 24-nt attR region. Conditions for PCR were as follows: denaturation for 3 min at 95° C.; profile for two first cycles: 1 min at 95° C., 30 sec at 50° C., 40 sec at 72° C.; profile for the last 25 cycles: 30 sec at 95° C., 30 sec at 54° C., 40 sec at 72° C.; final step: 5 min at 72° C.

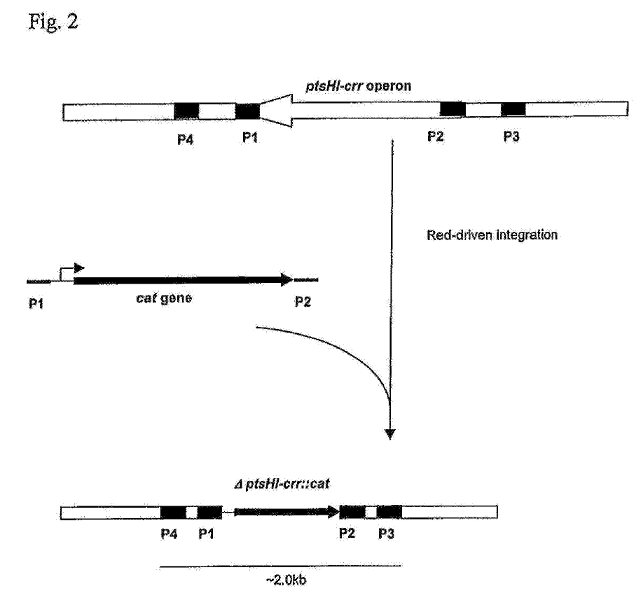

A 1699-bp PCR product (FIG. 2) was obtained and purified in agarose gel and was used for electroporation of the E. coli strain MG1655 (ATCC 700926), which contains the plasmid pKD46, the replication of which is temperature-sensitive. The plasmid pKD46 (Datsenko, K. A. and Wanner, B. L., Proc. Natl. Acad. Sci. USA, 2000, 97:12:6640-45) includes a 2,154 nucleotide DNA fragment of phage λ (nucleotide positions 31088 to 33241, GenBank accession no. J02459), and contains genes of the λ Red homologous recombination system (γ, β, exo genes) under the control of the arabinose-inducible P_{araB} promoter. The plasmid pKD46 is necessary for integration of the PCR product into the chromosome of strain MG1655. MG1655 can be obtained from American Type Culture Collection. (P.O. Box 1549 Manassas, Va. 20108, U.S.A.).

Electrocompetent cells were prepared as follows: E. coli MG1655/pKD46 was grown overnight at 30° C. in LB medium containing ampicillin (100 mg/l), and the culture was diluted 100 times with 5 ml of SOB medium (Sambrook et al, “Molecular Cloning: A Laboratory Manual, Second Edition”, Cold Spring Harbor Laboratory Press, 1989) containing ampicillin and L-arabinose (1 mM). The cells were grown with aeration at 30° C. to an OD₆₀₀ of ≈0.6 and then were made electrocompetent by concentrating 100-fold and washing three times with ice-cold deionized H₂O. Electroporation was performed using 70 μl of cells and ≈100 ng of the PCR product. Cells after electroporation were incubated with 1 ml of SOC medium (Sambrook et al, “Molecular Cloning: A Laboratory Manual, Second Edition”, Cold Spring Harbor Laboratory Press, 1989) at 37° C. for 2.5 hours and then were plated onto L-agar containing chloramphenicol (30 μg/ml) and grown at 37° C. to select Cm^{R} recombinants. Then, to eliminate the pKD46 plasmid, two passages on L-agar with Cm at 42° C. were performed and the resulting colonies were tested for sensitivity to ampicillin.

2. Verification of the ptsHI-crr Operon Deletion by PCR

The mutants without the ptsHI-crr operon and having the Cm resistance gene were verified by PCR. Locus-specific primers P3 (SEQ ID NO: 9) and P4 (SEQ ID NO: 10) were used in PCR for the verification. Conditions for PCR verification were as follows: denaturation for 3 min at 94° C.; profile for 30 cycles: 30 sec at 94° C., 30 sec at 54° C., 1 min at 72° C.; final step: 7 min at 72° C. The PCR product obtained in the reaction using the parental ptsHI-crr⁺ strain MG1655 as a template was ˜3.0 kbp in length. The PCR product obtained in the reaction using the cells of the mutant strain as a template was ˜2.0 kbp in length (FIG. 2). The mutant strain was named MG1655 Δ ptsHI-crr::cat.

3. Elimination of Cm Resistance Gene (Cat Gene) from the Chromosome of E. coli

MG1655-Δ ptsHI-crr::cat strain

The Cm resistance gene (cat gene) was deleted from the chromosome of the E. coli MG1655 Δ ptsHI-crr::cat strain using the int-xis system. For that purpose E. coli strain MG1655 ΔptsHI-crr::cat was transformed with plasmid pMWts-Int/Xis (WO 05/010175). Transformant clones were selected on LB-medium containing 100 μg/ml of ampicillin. Plates were incubated overnight at 30° C. Transformant clones were cured from the cat gene by spreading the separate colonies at 37° C. (at this temperature repressor CIts is partially inactivated and transcription of the int/xis genes is derepressed) followed by selection of Cm^{S}Ap^{R} variants. Elimination of the cat gene from the chromosome of the strain was verified by PCR. Locus-specific primers P3 (SEQ ID NO: 9) and P4 (SEQ ID NO: 10) were used in PCR for the verification. Conditions for PCR verification were as described above. The PCR product obtained in reaction using cells without the cat gene as a template was ˜0.4 kbp in length. Thus, the strain with the inactivated ptsHI-crr operon and missing the cat gene was obtained. This strain was named MG1655 A ptsHI-crr.

### Example 2

### Replacement of the Native Promoter Region of the araFGH Operon in E. coli with the Hybrid P_{L-tac} Promoter

To replace the native promoter region of the araFGH operon, a DNA fragment carrying a hybrid P_{L-tac} promoter and the chloramphenicol resistance marker (Cm^{R}) encoded by the cat gene was integrated into the chromosome of the E. coli MG1655 AptsHI-crr in place of the native promoter region by the method described by Datsenko K. A. and Wanner B. L. (Proc. Natl. Acad. Sci. USA, 2000, 97, 6640-6645) which is also called “Red-mediated integration” and/or “Red-driven integration”, and is also described in Example 1.

The hybrid P_{L-tac} promoter was obtained by PCR using the chromosomal DNA of E. coli strain B-3996P_{L-tac}xylE (PCT application WO2006043730) as the template, and primers P5 (SEQ ID NO 11 and P6 (SEQ ID NO: 12). PCR was conducted as described in Example 1.

The amplified DNA fragment was purified by agarose gel-electrophoresis, extracted using “GenElute Spin Columns” (“Sigma”, USA) and precipitated by ethanol. The obtained DNA fragment was used for electroporation and Red-mediated integration into the bacterial chromosome of the E. coli MG1655 ΔptsHI-crr/pKD46 as described in Example 1.

Colonies which grew within 24 h were tested for the presence of a Cm^{R} marker instead of the araFGH operon native promoter region by PCR using primers P7 (SEQ ID NO: 13) and

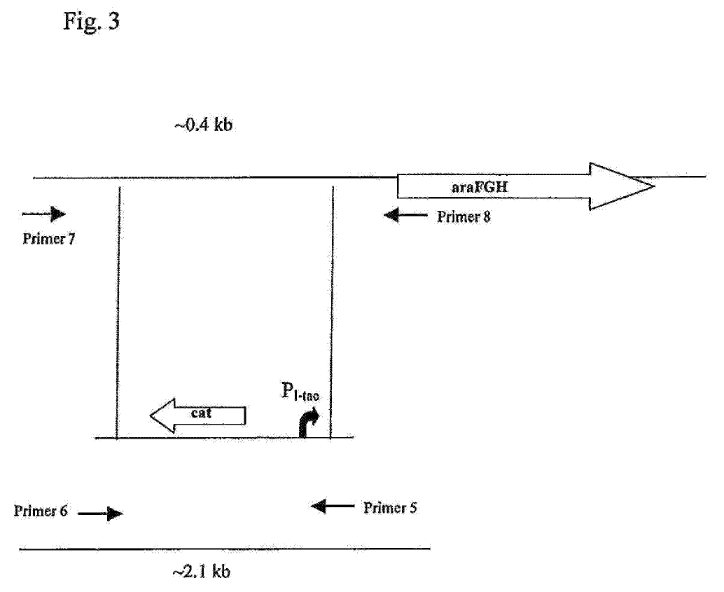

P8 (SEQ ID NO: 14). For this purpose, a freshly isolated colony was suspended in 20 μl water and then 11 of this suspension was used for PCR. PCR conditions were as described in Example 1. A few tested Cm^{R} colonies contained the desired ˜2.1 kb DNA fragment, confirming the presence of the hybrid P_{L-tac} promoter and Cm^{R} marker DNA instead of ˜0.4 kb araFGH operon native promoter region (see FIG. 3). One of the obtained strains was cured from the thermosensitive plasmid pKD46 by culturing at 37° C. and named E. coli MG1655 ΔptsHI-crr P_{L-tac}araFGH.

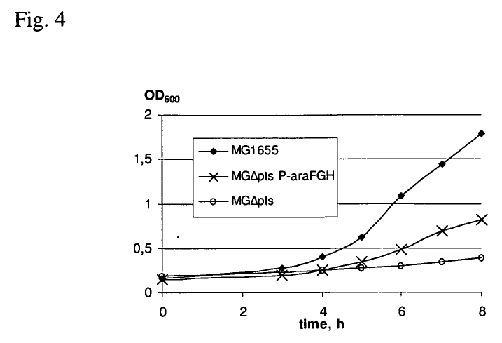

The ability to grow on the minimal Adams medium with glucose (4%) as a carbon source was checked for the three E. coli strains MG1655, MG1655ΔptsHI-crr, and MG1655ΔptsHI-crr P_{L-tac}araFGH. As seen in FIG. 4, E. coli MG1655AptsHI-crr did not grow well (μ˜0.06) on the minimal Adams medium containing glucose. Enhancing the araFGH operon expression significantly enhanced the growth characteristics of the recipient strains on the minimal Adams medium containing glucose.

### Example 3

### Effect of Enhancing the araFGH Operon Expression in the Strain Having a Disrupted PTS Transport System on L-Threonine Production

To disrupt the PTS transport system in the threonine-producing E. coli strain VKPM B-3996, the ptsH1-crr operon was inactivated. For that purpose DNA fragments from the chromosome of the above-described E. coli MG1655 ΔptsHI-crr::cat were transferred to the E. coli strain VKPM B-3996 by P1 transduction (Miller, J. H. Experiments in Molecular Genetics, Cold Spring Harbor Lab. Press, 1972, Plainview, N.Y.) to obtain the strain B-3996-Δ ptsHI-crr::cat.

The mutants without the ptsHI-crr operon and having the Cm resistance gene were verified by PCR. Locus-specific primers P3 (SEQ ID NO: 9) and P4 (SEQ ID NO: 10) were used in PCR for the verification. Conditions for PCR verification were as described above. The PCR product obtained in the reaction using the parental ptsHI-crr⁺ B-3996 strain as the template was ˜3.0 kbp in length. The PCR product obtained in the reaction using the mutant strain B-3996 ΔptsHI-crr::cat as the template was ˜2.0 kbp in length (FIG. 2).

The Cm resistance gene (cat gene) was deleted from the chromosome of the E. coli B-3996 ΔptsHI-crr::cat strain using the int-xis system. For that purpose, E. coli strain B-3996 ΔptsHI-crr::cat was transformed with plasmid pMWts-Int/X is (WO 2005 010175). Transformant clones were selected on the LB-medium containing 100 μg/ml of ampicillin. Plates were incubated overnight at 30° C. Transformant clones were cured from the cat gene by spreading the separate colonies at 37° C. (at this temperature repressor CIts is partially inactivated and transcription of the int/xis genes is derepressed) followed by selection of Cm^{S}Ap^{R} variants. Elimination of the cat gene from the chromosome of the strain was verified by PCR. Locus-specific primers P3 (SEQ ID NO: 9) and P4 (SEQ ID NO: 10) were used in PCR for the verification. Conditions for PCR verification were as described above. The PCR product obtained in reaction using cells without the cat gene as a template was ˜0.4 kbp in length. Thus, the threonine-producing strain with the inactivated ptsHI-crr operon and missing the cat gene was obtained. This strain was named B-3996ΔptsHI-crr.

For the purpose of enhancing the expression of the araFGH operon in E. coli B-3996AptsHI-crr, the native promoter of the araFGH operon was replaced with a hybrid P_{L-tac} promoter. For that purpose, DNA fragments from the chromosome of the above-described E. coli MG1655ΔptsHI-crr P_{L-tac}araFGH were transferred to the E. coli strain B-3996ΔptsHI-crr by P1 transduction (Miller, J. H. Experiments in Molecular Genetics, Cold Spring Harbor Lab. Press, 1972, Plainview, N.Y.) to obtain the E. coli strain B-3996-ΔptsHI-crr P_{L-tac}araFGH.

The deletion of the ptsHI-crr operon in the E. coli strain B-3996-ΔptsHI-crr P_{L-tac}araFGH was verified by PCR. Locus-specific primers P3 (SEQ ID NO: 9) and P4 (SEQ ID NO: 10) were used in PCR for the verification. Conditions for PCR verification were as described above. The PCR product obtained in the reaction using the strain B-3996-ΔptsHI-crr P_{L-tac}araFGH as the template was ˜0.4 kbp in length.

The substitution of the native promoter of the araFGH operon with hybrid P_{L-tac} promoter and Cm^{R} marker DNA in the E. coli strain B-3996-ΔptsHI-crr P_{L-tac}araFGH were verified by PCR. Locus-specific primers P7 (SEQ ID NO: 13) and P8 (SEQ ID NO: 14) were used in PCR for the verification. Conditions for PCR verification were as described above. The PCR product obtained in the reaction with the strain B-3996-ΔptsHI-crr P_{L-tac}araFGH as the template was ˜2.1 kbp in length.

Then, E. coli strains B-3996, B-3996-ΔptsHI-crr, and B-3996-ΔptsHI-crr P_{L-tac}araFGH were each cultivated at 37° C. for 18 hours in a nutrient broth, and 0.3 ml of each of the obtained cultures was inoculated into 3 ml of fermentation medium having the following composition in a 20×200 mm test tube and cultivated at 37° C. for 72 hours with a rotary shaker.

After cultivation, the accumulated amount of L-threonine in the medium was determined by paper chromatography using the following mobile phase: butanol:acetic acid:water=4:1:1 (v/v). A solution (2%) of ninhydrin in acetone was used as a visualizing reagent. The spot containing L-threonine was cut off, L-threonine was eluted in 0.5% water solution of CdCl₂, and the amount of L-threonine was estimated spectrophotometrically at 540 nm. The results of five tubes of fermentations are shown in Table 1.

The composition of the fermentation medium (g/l) was as follows:

| | |
|---|---|
| Glucose | 40.0 |
| (NH₄)₂SO₄ | 16.0 |
| K₂HPO₄ | 0.7 |
| MgSO₄•7H₂O | 1.0 |
| MnSO₄•5H₂O | 0.01 |
| FeSO₄•7H₂O | 0.01 |
| Thiamine hydrochloride | 0.002 |
| Yeast extract | 2.0 |
| L-isolucine | 0.01 |
| CaCO₃ | 33.0 |

MgSO₄.7H₂O and CaCO₃ were each sterilized separately.

TABLE 1

| Strain | OD₅₄₀ | Thr, g/l |
|---|---|---|
| B-3996 | 18.2 ± 0.7 | 18.9 ± 0.8 |
| B-3996ΔptsHI-crr | 0.85 ± 0.01 | 0.2 ± 0.01 |
| B-3996ΔptsHI-crr P_{L-tac}araFGH | 17.6 ± 0.6 | 19.5 ± 0.8 |

It can be seen from Table 1 that B-3996-ΔptsHI-crr P_{L-tac}araFGH caused the accumulation of a higher amount of L-threonine as compared with B-3996.

### Example 4

### Production of L-Lysine by E. coli AJ11442-P_{L-tac}araFGH

To test the effect of enhancing the araFGH operon on L-lysine production, DNA fragments coding for the arabinose transporter from the chromosome of the above-described E. coli MG1655-ΔptsHI-crr P_{L-tac}araFGH strain can be transferred to the lysine-producing E. coli strain AJ11442 by P1 transduction (Miller, J. H. Experiments in Molecular Genetics, Cold Spring Harbor Lab. Press, 1972, Plainview, N.Y.) to obtain strain AJ11442-P_{L-tac}araFGH. The strain AJ14442 was deposited at the National Institute of Bioscience and Human-Technology, Agency of Industrial Science and Technology (currently National Institute of Advanced Industrial Science and Technology, International Patent Organism Depositary, Tsukuba Central 6, 1-1, Higashi 1-Chome, Tsukuba-shi, Ibaraki-ken, 305-8566, Japan) on May 1, 1981 and received an accession number of FERM P-5084. Then, it was converted to an international deposit under the provisions of the Budapest Treaty on Oct. 29, 1987, and received an accession number of FERM BP-1543.

Both E. coli strains, AJ11442 and AJ11442-P_{L-tac}araFGH, can each be cultured in L-medium at 37° C., and 0.3 ml of each of the obtained cultures can be inoculated into 20 ml of the fermentation medium containing the required drugs in a 500-ml flask. The cultivation can be carried out at 37° C. for 16 h by using a reciprocal shaker at the agitation speed of 115 rpm. After the cultivation, the amounts of L-lysine and residual glucose in the medium can be measured by a known method (Biotech-analyzer AS210 manufactured by Sakura Seiki Co.). Then, the yield of L-lysine can be calculated relative to consumed glucose for each of the strains.

The composition of the fermentation medium (g/l) is as follows:

| | |
|---|---|
| Glucose | 40 |
| (NH₄)₂SO₄ | 24 |
| K₂HPO₄ | 1.0 |
| MgSO₄•7H₂O | 1.0 |
| FeSO₄•7H₂O | 0.01 |
| MnSO₄•5H₂O | 0.01 |
| Yeast extract | 2.0 |

The pH is adjusted to 7.0 by KOH and the medium is autoclaved at 115° C. for 10 min. Glucose and MgSO₄ 7H₂O are sterilized separately. CaCO₃ is dry-heat sterilized at 180° C. for 2 hours and added to the medium for a final concentration of 30 g/l.

### Example 5

### Production of L-Cysteine by E. coli JM15-P_{L-tac}araFGH

To test the effect of enhancing the araFGH operon on L-cysteine production, DNA fragments coding for the arabinose transporter from the chromosome of the above-described E. coli MG1655-ΔptsHI-crr P_{L-tac}araFGH strain can be transferred to the E. coli L-cysteine-producing strain JM15(ydeD) by P1 transduction (Miller, J. H. Experiments in Molecular Genetics, Cold Spring Harbor Lab. Press, 1972, Plainview, N.Y.) to obtain the strain JM15(ydeD)-P_{L-tac}araFGH.

E. coli strain JM15(ydeD) is a derivative of E. coli strain JM15 (U.S. Pat. No. 6,218,168) which can be transformed with DNA having the ydeD gene, which codes for a membrane protein, and is not involved in a biosynthetic pathway of any L-amino acid (U.S. Pat. No. 5,972,663). The strain JM15 (CGSC# 5042) can be obtained from The Coli Genetic Stock Collection at the E. coli Genetic Resource Center, MCD Biology Department, Yale University (http://cgsc.biology.yale.edu/).

Fermentation conditions for evaluation of L-cysteine production were described in detail in Example 6 of U.S. Pat. No. 6,218,168.

### Example 6

### Production of L-Leucine by E. coli 57-P_{L-tac}araFGH

To test the effect of enhancing the araFGH operon on L-leucine production, DNA fragments coding for the arabinose transporter from the chromosome of the above-described E. coli MG1655-ΔptsHI-crr P_{L-tac}araFGH strain can be transferred to the E. coli L-leucine-producing strain 57 (VKPM B-7386, U.S. Pat. No. 6,124,121) by P1 transduction (Miller, J. H. Experiments in Molecular Genetics, Cold Spring Harbor Lab. Press, 1972, Plainview, N.Y.) to obtain the strain 57-P_{L-tac}araFGH. The strain 57 has been deposited in the Russian National Collection of Industrial Microorganisms (VKPM) (Russia, 117545 Moscow, 1 Dorozhny proezd, 1) on May 19, 1997 under accession number VKPM B-7386.

Both E. coli strains, 57 and 57-P_{L-tac}araFGH, can each be cultured for 18-24 hours at 37° C. on L-agar plates. To obtain a seed culture, the strains can be grown on a rotary shaker (250 rpm) at 32° C. for 18 hours in 20×200-mm test tubes containing 2 ml of L-broth supplemented with 4% sucrose. Then, the fermentation medium can be inoculated with 0.21 ml of seed material (10%). The fermentation can be performed in 2 ml of a minimal fermentation medium in 20×200-mm test tubes. Cells can be grown for 48-72 hours at 32° C. with shaking at 250 rpm. The amount of L-leucine can be measured by paper chromatography (liquid phase composition: butanol-acetic acid-water=4:1:1).

The composition of the fermentation medium (g/1) (pH 7.2) is as follows:

| | |
|---|---|
| Glucose | 60.0 |
| (NH₄)₂SO₄ | 25.0 |
| K₂HPO₄ | 2.0 |
| MgSO₄•7H₂O | 1.0 |
| Thiamine | 0.01 |
| CaCO₃ | 25.0 |

Glucose and CaCO₃ are sterilized separately.

### Example 7

### Production of L-Histidine by E. coli Strain 80-P_{L-tac}araFGH

To test the effect of enhancing the araFGH operon on L-histidine production, DNA fragments coding for the arabinose transporter from the chromosome of the above-described E. coli MG1655-ΔptsHI-crr P_{L-tac}araFGH strain can be transferred to the histidine-producing E. coli strain 80 by P1 transduction (Miller, J. H. Experiments in Molecular Genetics, Cold Spring Harbor Lab. Press, 1972, Plainview, N.Y.) to obtain the strain 80-P_{L-tac}araFGH. The strain 80 was described in Russian patent 2119536 and deposited in the Russian National Collection of Industrial Microorganisms (Russia, 117545 Moscow, 1 Dorozhny proezd, 1) on Oct. 15, 1999 under accession number VKPM B-7270 and then converted to a deposit under the Budapest Treaty on Jul. 12, 2004.

Both E. coli strains, 80 and 80-P_{L-tac}araFGH, can each be cultured in L-broth for 6 h at 29° C. Then, 0.1 ml of each of the obtained cultures can be inoculated into 2 ml of fermentation medium in a 20×200-mm test tube and cultivated for 65 hours at 29° C. with shaking on a rotary shaker (350 rpm). After cultivation, the amount of histidine which accumulates in the medium can be determined by paper chromatography. The paper can be developed with a mobile phase consisting of n-butanol:acetic acid:water=4:1:1 (v/v). A solution of ninhydrin (0.5%) in acetone can be used as a visualizing reagent.

The composition of the fermentation medium (g/l) is as follows (pH 6.0):

| | |
|---|---|
| Glucose | 100.0 |
| Mameno (soybean hydrolysate) | 0.2 of as total nitrogen |
| L-proline | 1.0 |
| (NH₄)₂SO₄ | 25.0 |
| KH₂PO₄ | 2.0 |
| MgSO₄•7H₂0 | 1.0 |
| FeSO₄•7H₂0 | 0.01 |
| MnSO₄ | 0.01 |
| Thiamine | 0.001 |
| Betaine | 2.0 |
| CaCO₃ | 60.0 |

Glucose, proline, betaine and CaCO₃ are sterilized separately. The pH is adjusted to 6.0 before sterilization.

### Example 8

### Production of L-Glutamate by E. coli strain VL334thrC⁺-P_{L-tac}araFGH

To test the effect of enhancing the araFGH operon on L-glutamate production, DNA fragments coding for the arabinose transporter from the chromosome of the above-described E. coli MG1655-ΔptsHI-crr P_{L-tac}araFGH strain can be transferred to the E. coli L-glutamate-producing strain VL334thrC⁺ (EP 1172433) by P1 transduction (Miller, J. H. Experiments in Molecular Genetics, Cold Spring Harbor Lab. Press, 1972, Plainview, N.Y.) to obtain the strain VL334thrC⁺-P_{L-tac}araFGH. The strain VL334thrC⁺ has been deposited in the Russian National Collection of Industrial Microorganisms (VKPM) (Russia, 117545 Moscow, 1 Dorozhny proezd, 1) on Dec. 6, 2004 under the accession number VKPM B-8961 and then converted to a deposit under the Budapest Treaty on Dec. 8, 2004.

Both strains, VL334thrC⁺ and VL334thrC⁺-P_{L-tac}araFGH, can each be grown for 18-24 hours at 37° C. on L-agar plates. Then, one loop of the cells can be transferred into test tubes containing 2 ml of fermentation medium. The fermentation medium contains glucose (60 g/l), ammonium sulfate (25 μl), KH₂PO₄ (2 g/l), MgSO₄ (1 μl), thiamine (0.1 mg/ml), L-isoleucine (70 μg/ml), and CaCO₃ (25 μl). The pH is adjusted to 7.2. Glucose and CaCO₃ are sterilized separately. Cultivation can be carried out at 30° C. for 3 days with shaking. After the cultivation, the amount of L-glutamic acid produced can be determined by paper chromatography (liquid phase composition of butanol-acetic acid-water=4:1:1) with subsequent staining by ninhydrin (1% solution in acetone) and further elution of the compounds in 50% ethanol with 0.5% CdCl₂.

### Example 9

### Production of L-Phenylalanine by E. coli Strain AJ12739-P_{L-tac}araFGH

To test the effect of enhancing the araFGH operon on L-phenylalanine production, DNA fragments coding for the arabinose transporter from the chromosome of the above-described E. coli MG1655-ΔptsHI-crr P_{L-tac}araFGH strain can be transferred to the phenylalanine-producing E. coli strain AJ12739 by P1 transduction (Miller, J. H. Experiments in Molecular Genetics, Cold Spring Harbor Lab. Press, 1972, Plainview, N.Y.) to obtain the strain AJ12739-P_{L-tac}araFGH. The strain AJ12739 has been deposited in the Russian National Collection of Industrial Microorganisms (VKPM) (Russia, 117545 Moscow, 1 Dorozhny proezd, 1) on Nov. 6, 2001 under accession no. VKPM B-8197 and then converted to a deposit under the Budapest Treaty on Aug. 23, 2002.

Both strains, AJ12739-P_{L-tac}araFGH and AJ12739, can each be cultivated at 37° C. for 18 hours in a nutrient broth, and 0.3 ml of each of the obtained cultures can each be inoculated into 3 ml of a fermentation medium in a 20×200-mm test tube and cultivated at 37° C. for 48 hours with shaking on a rotary shaker. After cultivation, the amount of phenylalanine which accumulates in the medium can be determined by TLC. The 10×5-cm TLC plates coated with 0.11-mm layers of Sorbfil silica gel containing no fluorescent indicator (Stock Company Sorbpolymer, Krasnodar, Russia) can be used. The Sorbfil plates can be developed with a mobile phase consisting of propan-2-ol:ethylacetate:25% aqueous ammonia:water=40:40:7:16 (v/v). A solution of ninhydrin (2%) in acetone can be used as a visualizing reagent.

The composition of the fermentation medium (g/l) is as follows:

| | |
|---|---|
| Glucose | 40.0 |
| (NH₄)₂SO₄ | 16.0 |
| K₂HPO₄ | 0.1 |
| MgSO₄•7H₂O | 1.0 |
| FeSO₄•7H₂O | 0.01 |
| MnSO₄•5H₂O | 0.01 |
| Thiamine HCl | 0.0002 |
| Yeast extract | 2.0 |
| Tyrosine | 0.125 |
| CaCO₃ | 20.0 |

Glucose and magnesium sulfate are sterilized separately. CaCO₃ is dry-heat sterilized at 180° for 2 hours. The pH is adjusted to 7.0.

### Example 10

### Production of L-Tryptophan by E. coli Strain SV164 (pGH5)-P_{L-tac}araFGH

To test the effect of enhancing the araFGH operon on L-tryptophan production, DNA fragments coding for the arabinose transporter from the chromosome of the above-described E. coli MG1655-ΔptsHI-crr P_{L-tac}araFGH strain can be transferred to the tryptophan-producing E. coli strain SV164 (pGH5) by P1 transduction (Miller, J. H. Experiments in Molecular Genetics, Cold Spring Harbor Lab. Press, 1972, Plainview, N.Y.) to obtain the strain SV164(pGH5)-P_{L-tac}araFGH. The strain SV164 has the trpE allele, which encodes anthranilate synthase and is not subject to feedback inhibition by tryptophan. The plasmid pGH5 harbors a mutant serA gene, which encodes phosphoglycerate dehydrogenase and is not subject to feedback inhibition by serine. The strain SV164 (pGH5) was described in detail in U.S. Pat. No. 6,180,373 and European patent 0662143.

Both strains, SV164(pGH5)-P_{L-tac}araFGH and SV164(pGH5), can each be cultivated with shaking at 32° C. for 18 hours in 3 ml of nutrient broth supplemented with tetracycline (10 mg/l, marker of pGH5 plasmid). The obtained cultures (0.3 ml each) can each be inoculated into 3 ml of a fermentation medium containing tetracycline (10 mg/l) in 20×200-mm test tubes, and cultivated at 32° C. for 72 hours with a rotary shaker at 250 rpm. After cultivation, the amount of tryptophan which accumulates in the medium can be determined by TLC as described in Example 9.

The fermentation medium components are listed in Table 2, but should be sterilized in separate groups (A, B, C, D, E, F, and G), as shown, to avoid adverse interactions during sterilization.

TABLE 2

| Groups | Component | Final concentration, g/l |
|---|---|---|
| A | KH₂PO₄ | 1.5 |
| | NaCl | 0.5 |
| | (NH₄)₂SO₄ | 1.5 |
| | L-Methionine | 0.05 |
| | L-Phenylalanine | 0.1 |
| | L-Tyrosine | 0.1 |
| | Mameno (total N) | 0.07 |
| B | Glucose | 40.0 |
| | MgSO₄•7H₂O | 0.3 |
| C | CaCl₂ | 0.011 |
| D | FeSO₄•7H₂O | 0.075 |
| | Sodium citrate | 1.0 |
| E | Na₂MoO₄•2H₂O | 0.00015 |
| | H₃BO₃ | 0.0025 |
| | CoCl₂•6H₂O | 0.00007 |
| | CuSO₄•5H₂O | 0.00025 |
| | MnCl₂•4H₂O | 0.0016 |
| | ZnSO₄•7H₂O | 0.0003 |
| F | Thiamine HCl | 0.005 |
| G | CaCO₃ | 30.0 |
| H | Pyridoxine | 0.03 |

Group A had pH of 7.1, adjusted by NH₄OH. Each group was sterilized separately.

### Example 11

### Production of L-Proline by E. coli Strain 702ilvA-P_{L-tac}araFGH

To test the effect of enhancing the araFGH operon on L-proline production, DNA fragments coding for the arabinose transporter from the chromosome of the above-described E. coli MG1655-ΔptsHI-crr P_{L-tac}araFGH strain can be transferred to the proline-producing E. coli strain 702ilvA by P1 transduction (Miller, J. H. Experiments in Molecular Genetics, Cold Spring Harbor Lab. Press, 1972, Plainview, N.Y.) to obtain the strain 702ilvA-P_{L-tac}araFGH. The strain 702ilvA has been deposited in the Russian National Collection of Industrial Microorganisms (VKPM) (Russia, 117545 Moscow, 1 Dorozhny proezd, 1) on Jul. 18, 2000 under accession number VKPM B-8012 and then converted to a deposit under the Budapest Treaty on May 18, 2001.

Both E. coli strains, 702ilvA and 702ilvA-P_{L-tac}araFGH, can each be grown for 18-24 hours at 37° C. on L-agar plates. Then, these strains can be cultivated under the same conditions as in Example 8.

### Example 12

### Production of L-Arginine by E. coli Strain 382-P_{L-tac}araFGH

To test the effect of enhancing the araFGH operon on L-arginine production, DNA fragments coding for the arabinose transporter from the chromosome of the above-described E. coli MG1655-ΔptsHI-crr P_{L-tac}araFGH strain can be transferred to the arginine-producing E. coli strain 382 by P1 transduction (Miller, J. H. Experiments in Molecular Genetics, Cold Spring Harbor Lab. Press, 1972, Plainview, N.Y.) to obtain the strain 382-P_{L-tac}araFGH. The strain 382 has been deposited in the Russian National Collection of Industrial Microorganisms (VKPM) (Russia, 117545 Moscow, 1 Dorozhny proezd, 1) on Apr. 10, 2000 under accession number VKPM B-7926 and then converted to a deposit under the Budapest Treaty on May 18, 2001.

Both strains, 382-P_{L-tac}araFGH and 382, can each be cultivated with shaking at 37° C. for 18 hours in 3 ml of nutrient broth, and 0.3 ml of each of the obtained cultures were inoculated into 2 ml of a fermentation medium in 20×200-mm test tubes and cultivated at 32° C. for 48 hours on a rotary shaker.

After the cultivation, the amount of L-arginine which had accumulated in the medium can be determined by paper chromatography using the following mobile phase: butanol:acetic acid:water=4:1:1 (v/v). A solution of ninhydrin (2%) in acetone can be used as a visualizing reagent. A spot containing L-arginine can be cut out, L-arginine can be eluted with 0.5% water solution of CdCl₂, and the amount of L-arginine can be estimated spectrophotometrically at 540 nm.

The composition of the fermentation medium (g/l) is as follows:

| | |
|---|---|
| Glucose | 48.0 |
| (NH4)₂SO₄ | 35.0 |
| KH₂PO₄ | 2.0 |
| MgSO₄•7H₂O | 1.0 |
| Thiamine HCl | 0.0002 |
| Yeast extract | 1.0 |
| L-isoleucine | 0.1 |
| CaCO3 | 5.0 |

Glucose and magnesium sulfate are sterilized separately. CaCO₃ is dry-heat sterilized at 180° C. for 2 hours. The pH is adjusted to 7.0.

While the invention has been described in detail with reference to preferred embodiments thereof, it will be apparent to one skilled in the art that various changes can be made, and equivalents employed, without departing from the scope of the invention. All the cited references herein are incorporated as a part of this application by reference.

### INDUSTRIAL APPLICABILITY

According to the present invention, production of L-amino acids by a bacterium of the Enterobacteriaceae family can be enhanced.

1. An L-amino acid producing bacterium of the Enterobacteriaceae family, wherein said bacterium has been modified to enhance the expression of the araFGH operon.

2. The bacterium according to claim 1, wherein the expression of the araFGH operon is enhanced by modifying an expression control sequence of the araFGH operon so that the gene expression is enhanced or by increasing the copy number of the araFGH operon.

3. The bacterium according to claim 1, wherein said bacterium is selected from the group consisting of the genera Escherichia, Enterobacter, Erwinia, Klebsiella, Pantoea, Providencia, Salmonella, Serratia, Shigella, and Morganella.

4. The bacterium according to claim 1, wherein said operon encodes:
(A) a protein comprising the amino acid sequence of SEQ ID NO: 2 or a variant thereof;
(B) a protein comprising the amino acid sequence of SEQ ID NO: 4 or a variant thereof; and
(C) a protein comprising the amino acid sequence of SEQ ID NO: 6 or a variant thereof;
wherein said variants have the activity of the high-affinity L-arabinose transporter when said variants are combined together.

5. The bacterium according to claim 1, wherein said operon comprises:
(A) a DNA comprising the nucleotide sequence of nucleotides 1 to 990 in SEQ ID NO: 1, or a DNA which is able to hybridize to a sequence complementary to said sequence, or a probe prepared from said sequence under stringent conditions;
(B) a DNA comprising the nucleotide sequence of nucleotides 1 to 1515 in SEQ ID NO: 3, or a DNA which is able to hybridize to a sequence complementary to said sequence, or a probe prepared from said sequence under stringent conditions; and
(C) a DNA comprising the nucleotide sequence of nucleotides 1 to 990 in SEQ ID NO: 5, or a DNA which is able to hybridize to a sequence complementary to said sequence, or a probe prepared from said sequence under stringent conditions; and
wherein, said DNAs encode proteins which have an activity of the high-affinity L-arabinose transporter when said proteins are combined together.

6. The bacterium according to claim 5, wherein said stringent conditions comprise washing at 60° C. at a salt concentration of 1×SSC and 0.1% SDS, for approximately 15 minutes.

7. The bacterium according to claim 1, wherein said bacterium has been additionally modified to enhance the activity of glucokinase.

8. The bacterium according to claim 1, wherein said bacterium has been additionally modified to enhance the activity of xylose isomerase.

9. The bacterium according to claim 1, wherein said bacterium is an L-threonine producing bacterium.

10. The bacterium according to claim 9, wherein said bacterium has been additionally modified to enhance expression of a gene selected from the group consisting of:
the mutant thrA gene which codes for aspartokinase homoserine dehydrogenase I and is resistant to feedback inhibition by threonine;
the thrB gene which codes for homoserine kinase;
the thrC gene which codes for threonine synthase;
the rhtA gene which codes for a putative transmembrane protein;
the asd gene which codes for aspartate-β-semialdehyde dehydrogenase;
the aspC gene which codes for aspartate aminotransferase (aspartate transaminase); and
combinations thereof.

11. The bacterium according to claim 10, wherein said bacterium has been modified to increase expression of said mutant thrA gene, said thrB gene, said thrC gene, and said rhtA gene.

12. The bacterium according to claim 1, wherein said bacterium is an L-lysine producing bacterium.

13. The bacterium according to claim 1, wherein said bacterium is an L-histidine producing bacterium.

14. The bacterium according to claim 1, wherein said bacterium is an L-phenylalanine producing bacterium.

15. The bacterium according to claim 1 wherein said bacterium is an L-arginine producing bacterium.

16. The bacterium according to claim 1, wherein said bacterium is an L-tryptophan producing bacterium.

17. The bacterium according to claim 1, wherein said bacterium is an L-glutamic acid producing bacterium.

18. A method for producing an L-amino acid comprising cultivating the bacterium according to claim 1 in a culture medium which contains glucose as a carbon source, and isolating the L-amino acid from the culture medium.

19. The method according to claim 18, wherein said L-amino acid is selected from the group consisting of L-threonine, L-lysine, L-histidine, L-phenylalanine, L-arginine, L-tryptophan, and L-glutamic acid.
